(19) United States
(12) Patent Application Publication
(10) Pub. No.: US 2022/0276234 A1
(43) Pub. Date: Sep. 1, 2022

(54) POINT-OF-CARE IMMUNOASSAY FOR QUANTITATIVE SMALL ANALYTE DETECTION

(71) Applicant: Decimadx, LLC, Tampa, FL (US)

(72) Inventor: Phillip Gibbs, Tampa, FL (US)

(21) Appl. No.: 17/739,731

(22) Filed: May 9, 2022

Related U.S. Application Data
(63) Continuation-in-part of application No. 16/227,844, filed on Dec. 20, 2018.
Application No. 16/227,844 is a continuation of application No. 13/657,625, filed on Oct. 22, 2012, now abandoned.
(60) Provisional application No. 61/637,143, filed on Apr. 23, 2012.
(60) Provisional application No. 61/550,141, filed on Oct. 21, 2011.

(51) Int. Cl. G01N 33/543; G01N 33/558; C12N 15/10; C12N 15/115

(52) U.S. Cl. CPC G01N 33/54306; G01N 33/558; C12N 15/1048; C12N 15/115; G01N 2470/06; C40B 40/00

(57) ABSTRACT

Point-of-care assays for quantitatively measuring the amount of small analytes, such as opioids, tetrahydrocannabinol (“THC”), or hormones, in a biological sample are disclosed. The assays are capable of non-competitive detection of a small analyte using binding agents that selectively bind the analyte and capture agents that selectively bind a complex of the binding agent and analyte but do not bind either free binding agent or free analyte. The assay is capable of simultaneous diction of multiple analytes for multiplex analysis and quantitative control. Quantitative measurements are obtained by plotting results against a response surface calculated from a plurality of analyte standards and adjusted using internal controls.

### CROSS REFERENCE TO RELATED APPLICATIONS

This application is a continuation-in-part of U.S. application Ser. No. 16/227,844, filed Dec. 20, 2018, which is a continuation of U.S. application Ser. No. 13/657,625, filed Oct. 22, 2012, now abandoned, which claims benefit of U.S. Provisional Application No. 61/637,143, filed Apr. 23, 2012, and U.S. Provisional Application No. 61/550,141, filed Oct. 21, 2011, all of which are hereby incorporated herein by reference in their entirety.

### FIELD OF THE INVENTION

The invention is generally related to an assay method for detecting the presence of an analyte in a sample and devices and kits for performing the same.

### REFERENCE TO SEQUENCE LISTING

The Sequence Listing submitted on May 9, 2022, as a text file named “DECI_100_CON_CIP_ST25.txt,” created on May 9, 2022, and having a size of 12,291 bytes is hereby incorporated by reference.

### BACKGROUND OF THE INVENTION

Prescription opioid abuse and addiction are taking a rapidly growing toll on individuals, institutions, and businesses in the United States. It has been estimated that nearly 2.5 million individuals initiate the nonmedical use of prescription opioids each year, and incidence of prescription opioid abuse now exceeds that of many conventional street drugs, including cocaine and heroin. Opioid prescriptions can be misused by a wide range of methods. Patients may seek prescription opioids for pain symptoms that are real, exaggerated, or nonexistent, visiting multiple physicians and filling the prescriptions at multiple pharmacies, a practice known as “doctor shopping.” These prescriptions may then be misused by the patients themselves, diverted to family members or friends, or sold on the black market.

With prescription drug abuse on the rise, it is important for health care providers to have a point-of-care method of identifying patients that are misusing opioids and/or other prescription drugs before providing additional prescriptions. Drug tests are currently available for detecting opioids in urine, hair, saliva, or blood. However, these assays are either not suitable for point-of-care detection or are not sufficiently quantitative. The same issues apply to other types of illegal drugs such as tetrahydrocannabinol (“THC”), the active ingredient in marijuana.

There are other small analyte point-of-care needs outside of the drug abuse context. For example, an individual's ability to metabolize nicotine has been shown to negatively correlate with their ability to respond to nicotine treatment. Smokers with reduced nicotine metabolism have higher blood nicotine levels and compensate for this by smoking less. These individuals also demonstrate higher levels of cessation in transdermal nicotine therapy trials. Conversely, individuals with a normal metabolic rate tend to smoke more and have lower cessation rates. These normal metabolizers may be candidates for higher-dose nicotine replacement, which might potentially give rise to adverse effects in those with impaired nicotine metabolism. A need therefore exists for a point-of-care assay to measure nicotine metabolism in a subject.

Within the context of toxicity, heavy metals become toxic when they are not metabolized by the body and accumulate in the soft tissues. Heavy metal toxicity can result in damaged or reduced mental and central nervous function, lower energy levels, and damage to blood composition, lungs, kidneys, liver, and other vital organs. Long-term exposure may result in slowly progressing physical, muscular, and neurological degenerative processes that mimic Alzheimer's disease, Parkinson's disease, muscular dystrophy, and multiple sclerosis. Allergies are not uncommon and repeated long-term contact with some metals or their compounds may even cause cancer. For some heavy metals, toxic levels can be just above the background concentrations naturally found in nature. Therefore, testing is essential. Several analytical methods are available to analyze the level of heavy metals, such as lead, in biological samples. The most common methods employed are flame atomic absorption spectrometry (AAS), graphite furnace atomic absorption spectrometry (GFAAS), anode stripping voltammetry (ASV), inductively coupled plasma-atomic emission spectroscopy (ICP/AES), and inductively coupled plasma mass spectrometry (ICP/MS). However, these laboratory methods are labor-intensive, time-consuming, and expensive.

There are other needs for point-of-care assays that are not currently available for non-human uses, especially in the veterinary areas. For example, pregnancy checking in livestock and domestic animals requires obtaining blood samples and shipping of the sample to a lab to measure hormone levels to assess pregnancy, or conducting an ultrasound exam, which requires specialized training and equipment. It would be much less expensive and efficient if point-of-care assays were available for measurements of biological samples at the site of collection.

Enzyme-mediated immunoassays are frequently used as an initial evaluation drug/hormone testing, especially using samples. Such assays can test for numerous drugs or drug classes, can determine if a class of substances is present or absent, and typically show adequate sensitivity. However, these assays are not specific and fail to distinguish between different drugs of the same class. Christo, et al., Pain Physician, 14:123-143 (2011).

It is an object of the invention to provide a point-of-care assay for quantitatively measuring the amount of small analyte, such as a drug of abuse, heavy metal, or hormone, in a biological sample from a subject at the place of collection to provide immediate results.

It is also an object of the present invention to provide kits for a point-of-care assay for measuring the amount of small analyte in a biological sample.

### SUMMARY OF THE INVENTION

A point-of-care assay has been developed for quantitatively measuring the amount of a small analyte in a biological sample from a subject. The analytes may be organic, inorganic, or organometallic compounds, or metal ions. Exemplary analytes include drugs, metabolites, biologics such as hormones, toxins, and environmental contaminants.

The assay can be either a competitive or non-competitive assay. However, in preferred embodiments, the assay is a non-competitive immunoassay, which typically involves the use of a binding agent and a capture agent. Low molecular weight analytes are not large enough for simultaneous binding using routine reagents such as sandwich assays which rely on two antibodies recognizing different epitopes of an antigen. In some embodiments, the non-competitive assay involves the use of a “binding agent” that selectively binds the analyte, forming a “capture complex” of the binding agent and the analyte, and a “capture agent” that selectively binds the capture complex but not free analyte, forming a “sandwich complex.” Representative examples of capture agents that binds the capture complex but not free analyte are shown in SEQ ID Nos. 13 and 21-35. In these embodiments, the amount of sandwich complex is directly related to the amount of analyte in the sample. The assay is capable of simultaneous detection of multiple analytes for multiplex analysis and quantitative control.

The assay generally involves combining the biological sample with an assay fluid, a drug binding agent that specifically binds a drug analyte, a calibration/control analyte, and a calibration/control binding agent that specifically binds the calibration analyte. Exemplary binding agents and capture agents include antibodies, nucleic acid aptamers, and peptide aptamers that specifically bind analyte or capture complex, respectively. The binding agents are preferably linked to detectable labels, e.g., fluorescent labels, to facilitate detection of the sandwich complex. The capture agent may also be directly or indirectly linked to a detectable label to normalize detection parameters, e.g., light intensity for fluorescent labels. In some preferred embodiments, only the mobile element contains a label.

In some embodiments, the binding agent or capture agent is a nucleic acid aptamer beacon linked to a fluorophore and quencher pair such that quenching or unquenching occurs when the capture complex or sandwich complex is formed. In a preferred embodiment, the binding agent is an aptamer, and the capture agent is an antibody. In other preferred embodiments, the binding agent is an antibody and the capture agent is an aptamer having or containing a sequence selected from SEQ ID Nos. 13 and 21-35, or a derivative or mutant thereof having or containing a sequence that has between about 70% and about 100% sequence identity (a) to any one of SEQ ID Nos. 13 and 21-35, (b) to the variable sequence region of any one of SEQ ID Nos. 13 and 21-35, or (c) to the structure-switching region of any one of SEQ ID Nos. 13 and 21-35. These aptamers can used for detecting or quantifying cannabinoids such as THC, 11-hydroxy-THC (HTHC), and THC-COOH.

In some embodiments, fluorescent molecules on the binding agents and capture agents form a fluorescence/Förster resonance energy transfer (FRET) donor-acceptor pair. In FRET, energy from a molecular fluorophore (donor) is excited to a high-energy state and transferred to another fluorophore (acceptor) via intermolecular dipole-dipole coupling.

The assay is preferably a lateral flow immunoassay. Lateral flow immunoassays typically involve a membrane strip with an application point (e.g., a sample pad), an optional conjugate zone, a capture zone, and an absorption zone (e.g. wicking pad). A particularly preferred membrane strip is FUSION 5™ (Whatman Inc.), which can perform all the functions of a lateral flow strip on a single material. A biological sample, optionally combined with an assay fluid, is added to the application point at the proximal end of the strip, and the strip is maintained under conditions which allow the sample to transport by capillary action through the strip to and through the capture zone.

In some embodiments, the binding agents are added to the biological sample prior to administration to the application point of the membrane strip. In other embodiments, the sample migrates through the conjugate zone, where binding agents have been immobilized. The sample re-mobilizes the binding agents, and the analyte in the sample interacts with the binding agents to form capture complexes. The capture complexes then migrate into the capture zone where one or more capture agents have been immobilized. Excess reagents move past the capture lines and are entrapped in the wicking pad.

The capture agents are preferably coated onto or linked (using for example, covalent linkage) capture particles that are physically trapped within the membrane. The capture agents be conjugated directly to the membrane. The capture zone may be organized into one or more capture lines containing capture agents. In preferred embodiments, the capture zone contains a plurality of capture lines for multiplex analysis, i.e., detection of two or more analytes. In addition, the capture zone may contain one or more control capture lines for detecting the presence of control analyte. The control analyte can be a dilution control, i.e., an analyte such as creatine that is typically present in the biological sample at predictable concentrations. The control analyte may also be a reference analyte at a known concentration used to provide quantitative correlations between label detection and analyte amounts.

The assay may also involve the use of a sample collection apparatus that is not in fluid contact with the solid phase apparatus. The sample collection apparatus may contain the binding agents. In certain embodiments, the binding agents are evaporatively dried, vacuum-dried or freeze-dried in the sample collection apparatus.

Quantitative measurements may be obtained by plotting results against a response surface calculated from a plurality of analyte standards and adjusted using internal controls. For example, to determine the amount of analyte in the sample, the amount of sandwich complex in each capture line of the capture zone is assessed by measuring the detectable labels linked to the binding or capture agents.

In some embodiments, detectable label immobilized in or on the membrane (e.g., coated on capture particles trapped within the membrane) may be used to normalize detection parameters, e.g., light intensity for fluorescent labels. In these embodiments, the ratio of detectable label on binding agents to that of those immobilized in or on the membrane is preferably plotted against a response surface calculated from a plurality of analyte standards. In preferred embodiments, three or more internal standard analytes (needed to detect curvature) are detected concurrently with unknown analytes and used to adjust the predetermined response surface to minimize error for that particular assay run.

The disclosed lateral flow immunoassay provides a fast and accurate determination of the amount of a small analyte (e.g., drug, drug metabolite, heavy metal, or hormone) in a biological sample at the place of collection to provide immediate results.

Alternatively, the point-of-care assay described herein uses magnetic beads/particles, which are conjugated with the binding agent or the capture agent. Preferably, the magnetic beads/particles are conjugated with the capture agent, such as aptamers, which can detect immunocomplexes formed of analytes and their antibodies in solution. Preferably, the detectable label is linked to the free agent in solution, i.e., the agent not conjugated to the magnetic beads/particles. For example, if the capture agent is conjugated with the magnetic beads/particles, the binding agent is the free agent and is thus labeled with the detectable label, and vice versa. When there are multiple types of free agents in solution, e.g., multiple types of binding agents, the free agents can be labeled with a single type of detectable label for total analyte detection, or labeled with different types of detectable labels for simultaneous detection of a group of analytes.

Conventional lateral flow immunoassays usually have up to 12 detection lanes, whereas the use of magnetic beads/particles can allow for simultaneous detection of an even larger number of analytes. In some embodiments, this can be achieved by using either (a) magnetic beads/particles conjugated with different types of capture agents (e.g., aptamers), and/or (b) a mixture of different groups of magnetic beads/particles, wherein each group is conjugated with a distinct type of capture agent.

The use of magnetic beads/particles is advantageous for detection of a class of compounds, such as opioids or cannabinoids. When simultaneously detecting a group of structurally similar compounds, such as THC, 11-hydroxy-THC, and tetrahydrocannabinolic acid, the magnetic bead-based assays may simply require the use of a single antibody, which has septicity for all of the structurally similar compounds. The magnetic beads/particles can be conjugated with different types of aptamers, with each type of aptamers recognizing a distinct immunocomplex formed of a specific compound and its antibody.

### BRIEF DESCRIPTION OF THE DRAWINGS

FIGS. 1A and 1B are illustrations of a small analyte detection method using antibodies that specifically bind the analyte (binding agent), and DNA or RNA aptamers that specifically bind the antibody/analyte conjugate (metatype) (i.e., capture agent). In FIG. 1A, the antibody is shown bound to a fluorescent marker with an emission wavelength of Em2, and the aptamer is shown conjugated to an immobilized fluorescent particle with an emission wavelength of Em1. In FIG. 1B, the aptamer is shown conjugated to a fluorescent marker with an emission wavelength of Em2, and the antibody is shown conjugated to an immobilized fluorescent particle with an emission wavelength of Em1. The analyte is bound by the antibody, which is then captured by the aptamer. Excitation wavelengths Ex1 or Ex2 may then be used to detect fluorescent particle (as a control) and fluorescent marker, respectively.

FIG. 2A is an illustration of a small analyte detection method using antibodies that specifically bind the analyte (binding agent) and protein aptamers that specifically bind the antibody/analyte conjugate (metatype) (i.e., capture agents). In FIG. 2A, the antibody is shown bound to a fluorescent marker with an emission wavelength of Em2, and the aptamer is shown conjugated to an immobilized fluorescent particle with an emission wavelength of Em1. FIG. 2B is an illustration of small analyte detection method using protein aptamers that specifically bind the analyte (binding agent) and antibodies that specifically bind the aptamer/analyte conjugate (metatype) (i.e., capture agents). In FIG. 2B, the aptamer is shown bound to a fluorescent marker with an emission wavelength of Em2, and the antibody is shown conjugated to a fluorescent particle with an emission wavelength of Em1.

FIGS. 3A-3B are illustrations of a small analyte detection method using a DNA/RNA complex and an aptamer (FIG. 3A) or an antibody (FIG. 3B) that together bind analyte. The DNA/RNA complex is shown conjugated to a quenched fluorescent marker that is unquenched with an emission wavelength of Em2 when bound to analyte and aptamer (FIG. 3A) or antibody (FIG. 3B). The aptamer (FIG. 3A) and antibody (FIG. 3B) are shown conjugated to an immobilized fluorescent particle with an emission wavelength of Em1. FIG. 3C is an illustration of a small analyte detection method using antibodies that specifically bind the analyte (binding agent) and a DNA/RNA folding aptamer beacon that specifically bind the antibody/analyte conjugate (i.e., capture agent). The DNA/RNA complex is shown conjugated to an immobilized fluorescent particle with an emission wavelength of Em1 and also conjugated to a quenched fluorescent marker that is unquenched with an emission wavelength of Em2 when bound to analyte and antibody.

FIG. 4 is an illustration of a lateral flow device made from a membrane strip having an application point at the proximal end, followed by a conjugation zone, a capture zone, and an absorbent zone. The arrow shows the direction of lateral flow from the proximal to distal end. A plurality of capture lines are shown in the capture zone.

FIG. 5 is a scatter plot showing the specificity of the THC.5B7 and THC2.B9 antibodies for HTHC over THC at varying concentrations of HTHC and THC. Less reagent conversion indicates the displacement of the conjugate by the metabolite, and less overall higher affinity of the antibody for the metabolite. The HTHC and THC binding affinity was expressed as a HTHC/THC ratio. Values greater than 1 indicated that THC is preferred over HTHC, and values less than 1 indicated that HTHC is preferred.

FIGS. 6A and 6B are representative electropherograms of aptamer samples generated via CE SELEX for aptamers with B7-HTHC specificity (FIG. 6A) and aptamers with B9-THC specificity (FIG. 6B).

FIG. 7 is a representative electropherogram of the B7-HTHC samples.

FIG. 8 is a bar graph showing the percent of the library containing aptamers with more than 85 bases following selection of structure switching aptamers (SSAs).

FIGS. 9A-9D are graphical representations of the secondary structures of several of the candidate aptamers. The structure switching region extend from base 34 to base 45. FIG. 9A is the secondary structure of the aptamer contained in SEQ ID NO:6. FIG. 9B is the secondary structure of the aptamer contained in SEQ ID NO:31. FIG. 9C is the secondary structure of the aptamer contained in SEQ ID NO:32. FIG. 9D is the secondary structure of the aptamer contained in SEQ ID NO:33.

FIGS. 10A and 10B are electropherograms showing the peak shifts associated with the introduction of the immune complex in aptamer B7C1.

FIG. 11 is a qPCR amplification curve showing the binding of Aptamer B7C4 with the immune complex B7-HTHC, the immune complex B7-THC, the B7 antibody alone, and a negative control. Theoretically, every additional cycle indicates 2 times fewer bound aptamer molecules.

FIG. 12 is a graphical representation of the recombination steps during digestion and reshuffling of the library of B7-HTHC aptamer derivatives.

FIG. 13 is a bar graph showing the absorption signal from the sandwich type ELISA assay at different concentrations of HTHC or THC. FIG. 13 consolidates the results from two experiments, one in which HTHC is present (HTHC+) and a second experiment in which THC is absent (THC−).

FIG. 14 is a graphical representation of the most populous secondary structure identified after initial filtering of the NGS data.

FIGS. 15A-15E are graphical representations of the secondary structures of representative DNA aptamers obtained from aptamer selection and optimization. FIG. 15A represents the parent aptamer, B7C4-HTHC (SEQ ID NO:13); FIG. 15B is a representative aptamer from family 1 and is the secondary structure of the aptamer contained in SEQ ID NO:34; FIG. 15C is a representative aptamer from family 2 and is the secondary structure of the aptamer contained in SEQ ID NO:26; FIG. 15D is a representative aptamer from family 3 and is the secondary structure of the aptamer contained in SEQ ID NO:28; and FIG. 15E is the secondary structure of the aptamer with the highest number of mutations compared to the parent aptamer and is the secondary structure of the aptamer contained in SEQ ID NO:17.

FIGS. 16A and 16B are bar graphs showing the normalized absorption signal from the sandwich type ELISA assays for selected aptamers. In the ELISA assays, the antibody of HTHC, i.e., B7, was coated onto the walls of a microtiter plate. The aptamers were added either separately from (FIG. 16A) or simultaneously with (FIG. 16B) HTHC.

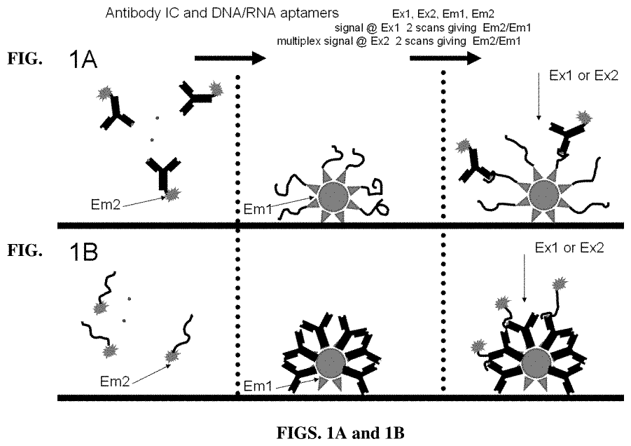

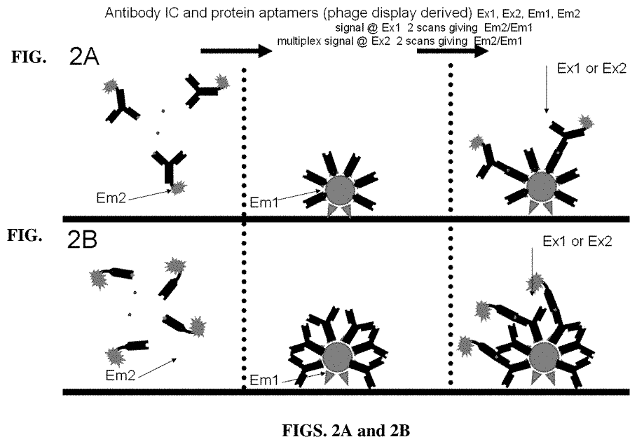

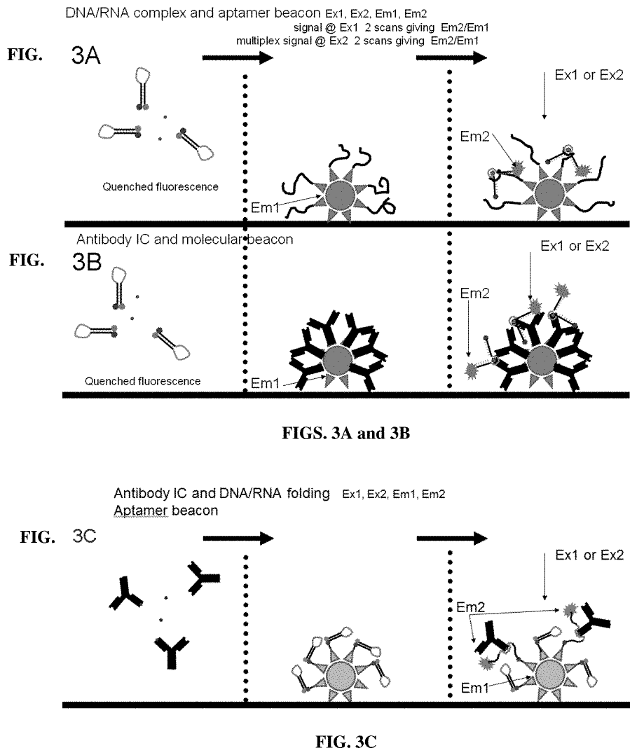

### DETAILED DESCRIPTION OF THE INVENTION

A point-of-care assay is disclosed that can be used to quantitatively measure one or more small analytes (e.g., drug, drug metabolite, heavy metal, or hormone) in a biological sample from a patient or a domestic animal or livestock at the place of collection. In particular, the point-of-care assay allows a physician to determine a subject's drug, drug metabolite, heavy metal, and/or hormone levels prior to prescribing any medication. In preferred embodiments, this assay can be done within 1 hour, preferably within 30 minutes, more preferably within 10 minutes of obtaining the biological sample.

### I. Definitions

The term “assay” refers to an in vitro procedure for analyzing a sample to determine the presence, absence, or quantity of one or more analytes of interest.

The terms “control” and “calibration” as used in connection with analytes, are used interchangeably to refer to analytes used as internal standards.

The term “analyte” refers to a chemical substance of interest that is a potential constituent of a biological sample and is to be analyzed by an assay.

The term “small analyte” refers to an analyte that is too small to be specifically bound by two antibodies that are specific for the analyte. For example, a small analyte may have a molecular weight of less than 2,000 Daltons, more preferably less than 1,500 Daltons, most preferably less than 1,000 Daltons. The small molecule can be a hydrophilic, hydrophobic, or amphiphilic compound.

The term “opioid” refers to a chemical that works by binding to opioid receptors. The term includes natural opiates as well as synthetic and semi-synthetic opioids.

The term “opioid metabolite” refers to a product of opioid metabolism in the patient.

The term “heavy metal” refers to a metal with a specific gravity that is at least five times the specific gravity of water.

A “lateral flow” assay is a device intended to detect the presence (or absence) of a target analyte in sample in which the test sample flows along a solid substrate via capillary action.

The term “membrane” as used herein refers to a solid substrate with sufficient porosity to allow movement of antibodies or aptamers bound to analyte by capillary action along its surface and through its interior.

The term “membrane strip” or “test strip” refers to a length and width of membrane sufficient to allow separation and detection of analyte.

The term “application point” is the position on the membrane where a fluid can be applied.

The term “binding agent” refers to a compound that specifically binds to an analyte. The term “capture agent” refers to an immobilized compound that selectively binds analyte complexed with binding agent (capture complex) or free binding agent (as a control). The capture agent may be conjugated to an immobilized capture particle. Binding agents and capture agents may be linked (directly or indirectly) to a detectable label. A binding agent is indirectly linked to a detectable label if it is bound to a particle that is directly linked to the detectable label. Binding agents and capture agents include antibodies, nucleic acid aptamers, and peptide aptamers.

The term “capture complex” refers to a complex formed by the specific binding of a binding agent to an analyte. The capture complex is immobilized for detection when captured by an immobilized capture agent.

The term “sandwich complex” refers to a complex formed by the specific binding of an immobilized capture agent to a binding agent and an analyte.

The term “immobilized” refers to chemical or physical fixation of an agent or particle to a location on or in a substrate, such as a membrane. For example, capture agents may be chemically conjugated to a membrane, and particles coated with capture agents may be physically trapped within a membrane.

The term “capture particle” refers to a particle coated with a plurality of capture agents. In preferred embodiments, the capture particle is immobilized in a defined capture zone.

The term “capture zone” refers to a point on a membrane strip at which one or more capture agents are immobilized.

The term “sandwich assay” refers to a type of immunoassay in which the analyte is bound between a binding agent and a capture agent. The capture agent is generally bound to a solid surface (e.g., a membrane or particle), and the binding agent is generally labeled.

The term “antibody” refers to intact immunoglobulin molecules, fragments or polymers of immunoglobulin molecules, single chain immunoglobulin molecules, human or humanized versions of immunoglobulin molecules, and recombinant immunoglobulin molecules, as long as they are chosen for their ability to bind an analyte.

The term “aptamer” refers to an oligonucleic acid or peptide molecule that binds to a specific target molecule. Aptamers are generally selected from a random sequence pool. The selected aptamers are capable of adapting unique tertiary structures and recognizing target molecules with high affinity and specificity.

A “nucleic acid aptamer” is an oligonucleic acid that binds to a target molecule via its conformation. A nucleic acid aptamer may be constituted by DNA, RNA, or a combination thereof. Nucleic acid aptamers are typically engineered using SELEX (systematic evolution of ligands by exponential enrichment).

A “peptide aptamer” is a combinatorial peptide molecule with a randomized amino acid sequence that is selected for its ability to bind a target molecule. Peptide aptamers are typically selected from combinatorial peptide libraries using yeast two-hybrid or phage display assays.

The term “metatype” refers to the analyte-binding site of a binding agent when bound to analyte. The term “idiotype” refers to the analyte binding site of a binding agent free of its analyte.

The term “anti-metatype” refers to a binding agent that selectively recognizes a binding agent-analyte complex (metatype) but lacks specificity for either free analyte or free binding agent. The term “anti-idiotype” refers to a binding agent that selectively recognizes the analyte binding site of another binding agent.

The term “specifically binds” or “selectively binds” refers to a binding reaction which is determinative of the presence of the analyte in a heterogeneous population. Generally, a first molecule that “specifically binds” a second molecule has an affinity constant (Kₐ) greater than about 10⁵ M⁻¹ (e.g., 10⁶ M⁻¹, 10⁷ M⁻¹, 10⁸ M⁻¹, 10⁹ M⁻¹, 10¹⁰ M⁻¹, 10¹¹ M⁻¹, and 10¹² M⁻¹ or more) with that second molecule.

The term “detectable label” refers to any moiety that can be selectively detected in a screening assay. Examples include radiolabels, (e.g., ³H, ¹⁴C, ³⁵S, ¹²⁵I, ¹³¹I), affinity tags (e.g., biotin/avidin or streptavidin), binding sites for antibodies, metal binding domains, epitope tags, fluorescent or luminescent moieties (e.g., fluorescein and derivatives, green fluorescent protein (GFP), rhodamine and derivatives, lanthanides), colorimetric probe, and enzymatic moieties (e.g., horseradish peroxidase, β-galactosidase, β-lactamase, luciferase, alkaline phosphatase).

The term “biological sample” refers to a tissue (e.g., tissue biopsy), organ, cell, cell lysate, or body fluid from a subject. Non-limiting examples of body fluids include blood, urine, plasma, serum, tears, lymph, bile, cerebrospinal fluid, interstitial fluid, aqueous or vitreous humor, colostrum, sputum, amniotic fluid, saliva, anal and vaginal secretions, perspiration, semen, transudate, exudate, and synovial fluid.

A “sample collection apparatus,” as used herein, refers to an apparatus that can be used for collection of a biological sample or into which a collected biological sample can be deposited or stored.

“Not in fluid contact,” as used herein, indicates that fluid will not flow passively from the sample collection apparatus onto/into application point. For example, physical separation or separation by a physical component can be used.

### II. Point-of-Care Assay

A rapid, reliable, sensitive, qualitative, and quantitative point-of-care assay was developed to quantitatively measure small analytes, such as hormones, heavy metals, drugs, or drug metabolites, in a biological sample from a patient, including human and veterinary subjects. The point-of-care assay can be used in combination with binding agents and capture agents that specifically bind drug, drug metabolites, heavy metals, or hormones.

There may be specialized examples where aptamers (employed alone) have been shown to recognize small molecules, however, in general, the binding affinity is poor and ability to evolve aptamers against all small molecule targets of interest has proved elusive. Jayasena, Clin. Chem., 45:1628-50 (1999). This is likely due to the relative lack of cooperative binding opportunities presented in small molecule targets and aptamers lack the more complex binding pocket of antibodies. Antibodies on the other hand, have a much richer structural pocket to evolve binding based on hydrophobic, ionic, and steric interactions, but however, present with problems of cross-reactivity, especially where small molecules are concerned. The problem of cross-reactivity in antibodies is apparent when looking at the opiate structures since molecules are so structurally similar (differing by as little as one side group). Since antibodies are typically selected by the host organism immune system to bind with the highest affinity and this often times result in antibodies targeting structurally similar motifs, the observed problems of cross reactivity are observed in opiates. Additionally, raising antibodies in vivo against the desired immunocomplex is very difficult and impractical in almost all cases. In contrast, aptamers can be evolved against the target immunocomplexes under nearly identical conditions for the ultimate immunoassay.

Aptamers for proteins generally exhibit higher affinities, because of the presence of larger complex areas with structures rich in hydrogen-bond donors and acceptors. Affinities in the nanomolar and sub nanomolar range have been measured for aptamers against different proteins. Mascini, et al., Angew. Chem. Int. Ed., 51:1316-1332 (2012). While not being bound by theory, the sandwich assays described herein “converts” small molecules which are in general poor targets for aptamers into proteins targets which are much better (i.e., nanomolar affinities compared to micromolar) targets. An immunocomplex of antibody and target molecule, for example, represent a much richer target for aptamer binding. Evolving aptamers against the much richer binding target of the immunocomplex between antibodies and the target molecules is a much more generalizable strategy (i.e., no special label required for each target molecule for immobilization as the antibodies already present a generalizable handle for the required immobilization). As such, tight binders to the complex are much easier to evolve and in the case where the structurally similar motif is buried in the antibody pocket, the external facing part of the molecule will likely contain the differentiating side group structure which can be recognized by the aptamer and hence lead to specific recognition of the desired immunocomplex as opposed to cross reactive immunocomplexes.

The point-of-care assay described herein is preferably a lateral flow immunoassay. In some embodiments, the assay involves the use of a sample collection apparatus that is not in fluid contact with the solid phase apparatus.

Alternatively, the point-of-care assay described herein uses magnetic beads/particles, which are conjugated with the binding agent or the capture agent. Preferably, the magnetic beads/particles are conjugated with the capture agent, such as aptamers, which can detect immunocomplexes formed of analytes and their antibodies in solution. Preferably, the detectable label is linked to the free agent in solution, i.e., the agent not conjugated to the magnetic beads/particles. For example, if the capture agent is conjugated with the magnetic beads/particles, the binding agent is the free agent and is thus labeled with the detectable label, and vice versa. When there are multiple types of free agents in solution, e.g., multiple types of binding agents, the free agents can be labeled with a single type of detectable label for total analyte detection or labeled with different types of detectable labels for simultaneous detection of a group of analytes.

Conventional lateral flow immunoassays usually have up to 12 detection lanes, whereas the use of magnetic beads/particles can allow for simultaneous detection of a larger number of analytes. In some embodiments, this can be achieved by using either (a) magnetic beads/particles conjugated with different types of capture agents (e.g., aptamers), and/or (b) a mixture of different groups of magnetic beads/particles, wherein each group is conjugated with a distinct type of capture agent.

The use of magnetic beads/particles is advantageous for detection of a class of compounds, such as opioids or cannabinoids. When simultaneously detecting a group of structurally similar compounds, such as THC, 11-hydroxy-THC, and tetrahydrocannabinolic acid, the magnetic bead-based assays may simply require the use of a single antibody, which has specificity for all the structurally similar compounds. The magnetic beads/particles can be conjugated with different types of aptamers, with each type of aptamers recognizing a distinct immunocomplex formed of a specific compound and its antibody.

A. Small Analytes to be Detected

Analytes which can be detected using the point-of-care assay described herein include, but are not limited to drugs, or drug metabolites, hormones, and heavy metals. In some embodiments, the analytes are drugs of abuse and metabolites thereof.

i. Drugs and Drug Metabolites

The assay can be used to quantitatively determine the levels of drugs, for example, drugs with potential for abuse, in a biological sample. Exemplary drugs and drug metabolites are described below. In some embodiments, the assay is semi-quantitative e.g., a test strip where the different opiates and metabolites are indicated by separate colors and analyzed by visual inspection.

1. Opioids

Exemplary opioids that can be detected using the quantitative point-of-care assay include morphine, codeine, thebaine, heroin, hydromorphone, hydrocodone, oxycodone, oxymorphone, desomorphine, nicomorphine, propoxyphene, dipropanoylmorphine, benzylmorphine, ethylmorphine, buprenorphine, fentanyl, pethidine, meperidine, methadone, tramadol, dextropropoxyphene, or analogues or derivatives thereof. For example, oxycodone (OxyContin®) is an opioid analgesic medication synthesized from opium-derived thebaine. Percocet is a combination of oxycodone and acetaminophen (paracetamol). Vicodin is a combination of hydrocodone and acetaminophen (paracetamol). In preferred embodiments, the assay quantitatively measures oxycodone, hydrocodone, or a combination thereof.

Exemplary opioid metabolites that can be detected using the disclosed quantitative lateral flow immunoassay are shown in Table 1.

TABLE 1
Opioid Metabolites

| | Key metabolizing | |
|---|---|---|
| Opioid | enzyme(s) | Major metabolites |
| Buprenorphine | CYP3A4 | Norbuprenorphine, |
| | | glucuronides |
| Codeine | CYP3A4, 2D6 | Morphine, glucuronides |
| Fentanyl | CYP3A4 | Norfentanyl |
| Hydrocodone | CYP3A4, 2D6 | Hydromorphone, |
| | | norhydrocodone |
| Hydromorphone | UGT1A3, 2B7 | Glucuronides |
| Meperidine | CYP3A4, 2B6, 2C19 | Normeperidine |
| Methadone | CYP2B6 | EDDP |
| Morphine | UGT2B7 | Glucuronides |
| Oxycodone | CYP3A4, 2D6 | Noroxycodone, oxymorphone |
| Oxymorphone | UGT2B7 | 6-OH-oxymorphone, |
| | | oxymorphone-3-glucuronide |
| Propoxyphene | CYP3A4 | Norpropoxyphene |
| Tramadol | CYP2D6 | O-desmethyl tramadol |

EDP = 2-Ethyl-1,5-dimethyl-3,3-diphenylpyrrolinium.

2. THC (Marijuana) and Cannabinoids

In the cannabis plant, THC occurs mainly as tetrahydrocannabinol carboxylic acid (THC-COOH). Geranyl pyrophosphate and olivetolic acid react, catalyzed by an enzyme to produce cannabigerolic acid, which is cyclized by the enzyme THC acid synthase to give THC-COOH. Over time, or when heated, THC-COOH is decarboxylated producing THC. THC is metabolized mainly to 11-OH-THC (11-hydroxy-THC, denoted as HTHC) by the human body. This metabolite is still psychoactive and is further oxidized to 11-nor-9-carboxy-THC (THC-COOH). More than 100 metabolites in humans and animals can be identified, but 11-OH-THC and THC-COOH are the dominating metabolites.

Metabolism occurs mainly in the liver by cytochrome P450 enzymes CYP2C9, CYP2C19, and CYP3A4. More than 55% of THC is excreted in the feces and approximately 20% in the urine. The main metabolite in urine is the ester of glucuronic acid and THC-COOH and free THC-COOH. In the feces, mainly 11-OH-THC is detected.

THC, 11-OH-THC (HTHC), and THC-COOH can be detected and quantified in blood, urine, hair, oral fluid or sweat. The concentrations obtained from such analyses can often be helpful in distinguishing active from passive use or prescription from illicit use, the route of administration (oral versus smoking), elapsed time since use and extent or duration of use.

Exemplary cannabinoids include Cannabichromenes such as Cannabichromene (CBC), Cannabichromenic acid (CBCA), Cannabichromevarin (CBCV) and Cannabichromevarinic acid (CBCVA); Cannabicyclols such as Cannabicyclol (CBL), Cannabicyclolic acid (CBLA) and Cannabicyclovarin (CBLV); Cannabidiols such as Cannabidiol (CBD), Cannabidiol monomethylether (CBDM), Cannabidiolic acid (CBDA), Cannabidiorcol (CBD-C1), Cannabidivarin (CBDV) and Cannabidivarinic acid (CBDVA); Cannabielsoins such as Cannabielsoic acid B (CBEA-B), Cannabielsoin (CBE) and Cannabielsoin acid A (CBEA-A); Cannabigerols such as Cannabigerol (CBG), Cannabigerol monomethylether (CBGM), Cannabigerolic acid (CBGA), Cannabigerolic acid monomethylether (CBGAM), Cannabigerovarin (CBGV) and Cannabigerovarinic acid (CBGVA); Cannabinols and cannabinodiols such as Cannabinodiol (CBND), Cannabinodivarin (CBVD), Cannabinol (CBN), Cannabinol methylether (CBNM), Cannabinol-C2 (CBN-C2), Cannabinol-C4 (CBN-C4), Cannabinolic acid (CBNA), Cannabiorcool (CBN-C1) and Cannabivarin (CBV); Cannabitriols such as 10-Ethoxy-9-hydroxy-delta-6a-tetrahydrocannabinol, 8,9-Dihydroxy-delta-6a-tetrahydrocannabinol, Cannabitriol (CBT) and Cannabitriolvarin (CBTV); Delta-8-tetrahydrocannabinols such as Delta-8-tetrahydrocannabinol (A8-THC) and Delta-8-tetrahydrocannabinolic acid (A8-THCA); Delta-9-tetrahydrocannabinols such as Delta-9-tetrahydrocannabinol (THC), Delta-9-tetrahydrocannabinol-C4 (THC-C4), Delta-9-tetrahydrocannabinolic acid A (THCA-A), Delta-9-tetrahydrocannabinolic acid B (THCA-B), Delta-9-tetrahydrocannabinolic acid-C4 (THCA-C4), Delta-9-tetrahydrocannabiorcol (THC-C1), Delta-9-tetrahydrocannabiorcolic acid (THCA-C1), Delta-9-tetrahydrocannabivarin (THCV) and Delta-9-tetrahydrocannabivarinic acid (THCVA); as well as 10-Oxo-delta-6a-tetrahydrocannabinol (OTHC), Cannabichromanon (CBCF), Cannabifuran (CBF), Cannabiglendol, Cannabiripsol (CBR), Cannbicitran (CBT), Dehydrocannabifuran (DCBF), Delta-9-cis-tetrahydrocannabinol (cis-THC), Tryhydroxy-delta-9-tetrahydrocannabinol (triOH-THC) and 3,4,5,6-Tetrahydro-7-hydroxy-alpha-alpha-2-trimethyl-9-n-propyl-2,6-methano-2H-1-benzoxocin-5-methanol (OH-iso-HI-ICV) and analogs thereof.

3. Nicotine

As nicotine enters the body, it is distributed quickly through the bloodstream and crosses the blood-brain barrier reaching the brain within 10-20 seconds after inhalation. The elimination half-life of nicotine in the body is approximately two hours. The amount of nicotine absorbed by the body from smoking depends on many factors, including the types of tobacco, whether the smoke is inhaled, and whether a filter is used. For chewing tobacco, dipping tobacco, snus and snuff, which are held in the mouth between the lip and gum, or taken in the nose, the amount released into the body tends to be much greater than smoked tobacco.

Nicotine is metabolized in the liver by cytochrome P450 enzymes (mostly CYP2A6, and by CYP2B6). A major metabolite of nicotine that is excreted in the urine is cotinine, which is a reliable and necessary indicator of nicotine usage. Other primary metabolites include nicotine N′-oxide, nornicotine, nicotine isomethonium ion, 2-hydroxynicotine and nicotine glucuronide. Glucuronidation and oxidative metabolism of nicotine to cotinine are both inhibited by menthol, an additive to mentholated cigarettes, thus increasing the half-life of nicotine in vivo.

Nicotine (cotinine) can be quantified in blood, plasma, or urine to confirm a diagnosis of poisoning or to facilitate a medicolegal death investigation. Urinary or salivary cotinine concentrations are frequently measured for the purposes of pre-employment and health insurance medical screening programs. Careful interpretation of results is important, since passive exposure to cigarette smoke can result in significant accumulation of nicotine, followed by the appearance of its metabolites in various body fluids.

The CYP2A6 enzyme is genetically polymorphic with certain alleles predicting altered metabolic activity. As the primary enzyme for nicotine metabolism, variation in the metabolic activity of CYP2A6 has a significant effect on an individual's level of tobacco consumption. The reduced metabolism phenotype leads to higher blood/nicotine levels and smokers tend to compensate for this by smoking less. Conversely, individuals with increased metabolic rate tend to smoke more. Lower nicotine metabolism with CYP2A6 variants also influences smoking cessation, with slow metabolizers demonstrating higher levels of cessation in transdermal nicotine therapy trials. This may be due to the higher therapeutic doses of nicotine that the slow metabolizer sub-group obtains from comparable levels of transdermal nicotine treatment. Normal metabolizers have lower cessation rates probably because of current treatments failing to provide high enough levels of replacement blood nicotine. These normal metabolizers may be candidates for higher-dose nicotine replacement, which might potentially give rise to adverse effects in those with impaired nicotine metabolism.

The disclosed compositions and methods may be used to evaluate a patient's metabolism of nicotine. For example, nicotine levels can be quantified using the disclosed compositions and methods after a controlled dosage of nicotine is administered to a patient. This can in some embodiments involve allowing the subject to smoke a cigarette. In preferred embodiments, a nicotine patch or gum is given to the subject for a prescribed amount of time. The amount of nicotine or a metabolite thereof (e.g., cotinine) in a biological sample of the subject may then be monitored for rate of change.

4. Stimulants

Psychostimulants comprise a broad class of licit and illicit sympathomimetic drugs whose effects can include increased movement, arousal, vigilance, anorexia, vigor, wakefulness, and attention (Westfall and Westfall, 2006, Adrenergic agonists and antagonists, in Goodman & Gilman's The Pharmacological Basis of Therapeutics, 11th Edition, The McGraw-Hill Companies, New York). Some psychostimulants, especially at high doses and with a rapid route of administration, produce euphoria, a sense of power and confidence, and addiction, in certain susceptible individuals (Boutrel and Koob, 2004, Sleep 27:1181-1194).

Amphetamines are a class of stimulants based on the amphetamine structure. They include all derivative compounds which are formed by replacing, or substituting, one or more hydrogen atoms in the amphetamine core structure with substituents. Examples of substituted amphetamines includes amphetamine (itself), methamphetamine, ephedrine, cathinone, phentermine, mephentermine, bupropion, methoxyphenamine, selegiline, amfepramone, pyrovalerone, MDMA (ecstasy), and DOM (STP). Many drugs in this class work primarily by activating trace amine-associated receptor 1 (TAAR1); in turn, this causes reuptake inhibition and effluxion, or release, of dopamine, norepinephrine, and serotonin. An additional mechanism of some amphetamines is the release of vesicular stores of monoamine neurotransmitters through VMAT2, thereby increasing the concentration of these neurotransmitters in the cytosol, or intracellular fluid, of the presynaptic neuron.

Cocaine and its analogues are another class of stimulants. They usually maintain a benzyloxy connected to the carbon 3 of a tropane. Various modifications include substitutions on the benzene ring, as well as additions or substitutions in place of the normal carboxylate on the tropane 2 carbon. Various compounds with similar structure-activity relationships to cocaine that are not technically analogues have been developed as well. Exemplary cocaine analogues include stereoisomers of cocaine, 3β-phenyl ring substituted analogues, 2β-substituted analogues, N-modified analogues of cocaine, 3β-carbamoyl analogues, 3β-alkyl-3-benzyl tropanes, 6/7-substituted cocaine, 6-alkyl-3-benzyl tropanes, and piperidine homologues of cocaine. Examples of cocaine analogues can be found in Singh, Chemistry, Design, and Structure-Activity Relationship of Cocaine Antagonists, Chem. Rev., 2000, 100, 925-1024.

5. Central Nerve System Depressants

Central nerve system (CNS) depressants typically slow brain activity, which makes them useful for treating anxiety and sleep problems.

Common CNS depressants include barbiturates such as pentobarbital (NEMBUTAL®), benzodiazepines, and sleep medications such as eszopiclone (LUNESTA®), zaleplon (SONATA®), and zolpidem (AMBIEN®).

Benzodiazepines (BZD, BDZ, BZs), sometimes called “benzos”, are a class of psychoactive drugs whose core chemical structure is the fusion of a benzene ring and a diazepine ring. Benzodiazepines enhance the effect of the neurotransmitter gamma-aminobutyric acid (GABA) at the GABAA receptor, resulting in sedative, hypnotic (sleep-inducing), anxiolytic (anti-anxiety), anticonvulsant, and muscle relaxant properties. High doses of many shorter-acting benzodiazepines may also cause anterograde amnesia and dissociation. These properties make benzodiazepines useful in treating anxiety, insomnia, agitation, seizures, muscle spasms, alcohol withdrawal and as a premedication for medical or dental procedures. Exemplary benzodiazepines include brotizolam, midazolam, triazolam, alprazolam, estazolam, flunitrazepam, clonazepam, lormetazepam, lorazepam, nitrazepam, temazepam, diazepam, clorazepate, chlordiazepoxide, flurazepam, halazepam, prazepam, oxazepam, nimetazepam, adinazolam, climazolam, loprazolam, and derivatives thereof.

6. Other Drugs and Drug Metabolites

Hallucinogens are psychoactive agents, which can cause hallucinations, perceptual anomalies, and other substantial subjective changes in thoughts, emotion, and consciousness. The common types of hallucinogens are psychedelics, dissociatives, and deliriants. Although hallucinations are a common symptom of amphetamine psychosis, amphetamines are not considered hallucinogens, as they are not a primary effect of the drugs themselves. Exemplary hallucinogens include ketamine, LSD and other ergotamine derivatives, mescaline and other phenethylamines, PCP, psilocybin, salvia, DMT and other tryptamines, and ayahuasca. Psychedelics include serotonergics and cannabinoidergics. Serotonergics can be further divided into indoles/tryptamines (such as psilocybin, ergolines such as LSD, beta-carbolines, and complexly substituted tryptamines such as ibogaine) and phenethylamines such as mescaline.

γ-Hydroxybutyric acid (GHB), also known as 4-hydroxybutanoic acid, is a naturally occurring neurotransmitter and a psychoactive drug. It is a precursor to GABA, glutamate, and glycine in certain brain areas. It acts on the GHB receptor and is a weak agonist at the GABA_{B} receptor. Its prodrugs and analogs include 3-methyl-GHB, 4-methyl-GHB, 4-phenyl-GHB, 4-hydroxy-4-methylpentanoic acid (UMB68), γ-valerolactone (GVL), 1,4-butanediol diacetate (BDDA/DABD), methyl-4-acetoxybutanoate (MAB), ethyl-4-acetoxybutanoate (EAB), and γ-hydroxybutyraldehyde (GHBAL).

Additional drugs include mitragynine, 7-hydroxymitragynine, and derivatives thereof.

Additional drugs also include steroids such as nandrolone (OXANDRIN®), oxandrolone (ANADROL®), oxymetholone (ANADROL-50®), and testosterone cypionate (DEPO-TESTOSTERONE®).

Additional drugs also include methylphenidate, ethylphenidate, and ritalinic acid. Methylphenidate is a stimulant medication used to treat attention deficit hyperactivity disorder (ADHD) and narcolepsy. Ethylphenidate acts as both a dopamine reuptake inhibitor and norepinephrine reuptake inhibitor, meaning it effectively boosts the levels of the norepinephrine and dopamine neurotransmitters in the brain, by binding to, and partially blocking the transporter proteins that normally remove those monoamines from the synaptic cleft. Ritalinic acid is a substituted phenethylamine and an inactive major metabolite of the psychostimulant drugs methylphenidate and ethylphenidate.

Aripiprazole is an atypical antipsychotic. It is primarily used in the treatment of schizophrenia and bipolar disorder. Other uses include as an add-on treatment in major depressive disorder, tic disorders and irritability associated with autism. A metabolite of aripiprazole, OPC-3373, is much more water soluble and can partition to oral fluids better than the parent compound and a more commonly assayed metabolite dehydro aripiprazole. All the foregoing three compounds can be the analytes.

ii. Hormones for Detection of Pregnancy or Time of Ovulation in Animals

Unlike in humans, the hormonal cycles of domestic pets such as dogs and of livestock such as horses, cattle and swine are not as easily assayed and there are no point-of-care assays available. However, the reproductive levels of hormones indicating onset of ovulation, timing of breeding, and pregnancy are well understood by those skilled in the art and can be readily quantitated using a point of care immunoassay as described herein.

There are multiple hormones that help to regulate the estrus (heat) cycle and pregnancy in animals. These include estrogen, which stimulates the ovaries to produce eggs, luteinizing hormone (LH), which stimulates the ovaries to release the eggs, and progesterone, which maintains a pregnancy. Most mammals ovulate when the estrogen level in the blood is increasing. Dogs, however, ovulate when the estrogen level is declining, and the progesterone level is increasing. Progesterone levels and luteinizing hormone (LH) levels are the best indicators of when ovulation will take place and when is the best time to breed.

iii. Heavy Metal Ions

Heavy metals are toxic and persistent environmental contaminants. Unlike carbon-based contaminants that can be completely degraded to relatively harmless products, metal ions can be transformed in only a limited number of ways by biological or chemical remediation processes.

Heavy metals have a specific gravity that is at least five times the specific gravity of water. Some well-known toxic metallic elements with a specific gravity that is five or more times that of water are arsenic, cadmium, iron, lead, and mercury. Additional toxic heavy metals include antimony, bismuth, cerium, chromium, cobalt, copper, gallium, gold, manganese, nickel, platinum, silver, tellurium, thallium, tin, uranium, vanadium, and zinc.

Heavy metal toxicity can result in damaged or reduced mental and central nervous function, lower energy levels, and damage to blood composition, lungs, kidneys, liver, and other vital organs. Long-term exposure may result in slowly progressing physical, muscular, and neurological degenerative processes that mimic Alzheimer's disease, Parkinson's disease, muscular dystrophy, and multiple sclerosis. Allergies are not uncommon and repeated long-term contact with some metals, or their compounds may even cause cancer.

Small amounts of these elements are common in our environment and diet and are necessary for good health, but large amounts of any of them may cause acute or chronic toxicity. Heavy metals become toxic when they are not metabolized by the body and accumulate in the soft tissues. Heavy metals may enter the human body through food, water, air, or absorption through the skin when humans come into contact with the min agricultural, manufacturing, pharmaceutical, industrial, or residential settings. Industrial exposure is a common route of exposure for adults. Ingestion is the most common route of exposure in children. Children may develop toxic levels of heavy metals from the normal hand-to-mouth activity of small children who come in contact with contaminated soil or by actually eating objects that are not food (dirt or paint chips). Less common routes of exposure are during a radiological procedure, from inappropriate dosing or monitoring during intravenous (parenteral) nutrition, from a broken thermometer, or from a suicide or homicide attempt.

For some heavy metals, toxic levels can be just above the background concentrations naturally found in nature. Therefore, it is important to take protective measures against excessive exposure. For persons who suspect that they or someone in their household might have heavy metal toxicity, testing is essential. The most common methods employed are flame atomic absorption spectrometry (AAS), graphite furnace atomic absorption spectrometry (GFAAS), anode stripping voltammetry (ASV), inductively coupled plasma-atomic emission spectroscopy (ICP/AES), and inductively coupled plasma mass spectrometry (ICP/MS). However, these laboratory methods are labor-intensive, time-consuming, and expensive.

Antibody-based assays offer an alternative approach for metal ion detection. Immunoassays are quick, inexpensive, simple to perform, and reasonably portable; they can also be highly sensitive and selective. Sample analysis is one of the major costs in the remediation of a contaminated site, and studies have shown that the use of antibody-based assays can reduce analysis costs by 50% or more.

B. Binding Agents and Capture Reagents

Binding agents for use in the disclosed assays include any molecule that selectively binds opioid analytes or calibration analytes. In preferred embodiments, the binding agents are antibodies, such as monoclonal antibodies, or aptamers, such as nucleic acid or peptide aptamers.

i. Antibodies

Antibodies that can be used in the compositions and methods include whole immunoglobulin (i.e., an intact antibody) of any class, fragments thereof, and synthetic proteins containing at least the antigen binding variable domain of an antibody. The variable domains differ in sequence among antibodies and are used in the binding and specificity of each antibody for its specific antigen. However, the variability is not usually evenly distributed through the variable domains of antibodies. It is typically concentrated in three segments called complementarity determining regions (CDRs) or hypervariable regions both in the light chain and the heavy chain variable domains. The more highly conserved portions of the variable domains are called the framework (FR). The variable domains of native heavy and light chains each comprise four FR regions, largely adopting a beta-sheet configuration, connected by three CDRs, which form loops connecting, and in some cases forming part of, the beta-sheet structure. The CDRs in each chain are held together in proximity by the FR regions and, with the CDRs from the other chain, contribute to the formation of the antigen binding site of antibodies. Therefore, the disclosed antibodies contain at least the CDRs necessary to maintain DNA binding and/or interfere with DNA repair.

Fragments of antibodies which have bioactivity can also be used. The fragments, whether attached to other sequences or not, include insertions, deletions, substitutions, or other selected modifications of specific regions or amino acids residues, provided the activity of the fragment is not significantly altered or impaired compared to the non-modified antibody or antibody fragment.

Techniques can also be adapted for the production of single-chain antibodies specific to an antigenic protein of the present disclosure. Methods for the production of single-chain antibodies are well known to those of skill in the art. A single chain antibody can be created by fusing together the variable domains of the heavy and light chains using a short peptide linker, thereby reconstituting an antigen binding site on a single molecule. Single-chain antibody variable fragments (scFvs) in which the C-terminus of one variable domain is tethered to the N-terminus of the other variable domain via a 15 to 25 amino acid peptide or linker have been developed without significantly disrupting antigen binding or specificity of the binding. The linker is chosen to permit the heavy chain and light chain to bind together in their proper conformational orientation.

Divalent single-chain variable fragments (di-scFvs) can be engineered by linking two scFvs. This can be done by producing a single peptide chain with two VH and two VL regions, yielding tandem scFvs. ScFvs can also be designed with linker peptides that are too short for the two variable regions to fold together (about five amino acids), forcing scFvs to dimerize. This type is known as diabodies. Diabodies have been shown to have dissociation constants up to 40-fold lower than corresponding scFvs, meaning that they have a much higher affinity to their target. Still shorter linkers (one or two amino acids) lead to the formation of trimers (triabodies or tribodies). Tetrabodies have also been produced. They exhibit an even higher affinity to their targets than diabodies.

Suitable antibodies may be commercially available. For example, antibodies that specifically bind codeine (Abcam® #ab31202), heroin (Randox Life Sciences #PAS10133), morphine (Abcam® #ab1060, #ab23357), hydrocodone (Abbiotec™ #252375), hydromorphone (Abcam® #ab58932), oxycodone (Abcam® #ab30544), propoxyphene (Abcam® #ab50726), buprenorphine (Abcam® #ab31201), fentanyl (Abcam® #ab30729, #ab31323), pethidine (Novus Biologicals® #NBP1-41034), meperidine (Abcam® #ab59530), methadone (Abcam® #ab35799), and tramadol (Abcam® #ab58934) are commercially available. Antibodies that specifically bind cannabinoid such as THC, HTHC, THC-COOH, cannabidiol, and/or their derivatives or metabolites are also commercially available, such as anti-THC antibody ab30792 from Abcam, anti-THC antibody from ThermoFisher Scientific (catalog #: PA1-75456), anti-THC antibody C29 from Santa Cruz (catalog #: sc-58054) and THC.5B7 and THC2.B9 antibodies (Bioventrix). Antibodies that specifically bind stimulants such as amphetamine and cocaine are also commercially available, such as anti-amphetamine antibody from LifeSpan BioSciences, Inc. (catalog #: LS-C55839-500), anti-amphetamine antibody from MyBioSource.com (catalog #: MBS319618), anti-cocaine antibody from LifeSpan BioSciences, Inc. (catalog #: LS-C85770-1), and anti-benzodiazepine antibody from LifeSpan BioSciences, Inc. (catalog #: LS-C194300-1).

Several antibodies have been reported with the ability to bind heavy metals. Monoclonal antibodies directed toward mercuric ions have been generated by immunization of animals with a glutathione-Hg derivative (Wylie et al., Proc. Natl. Acad. Sci. USA 89:4104-4108 (1992)). Recombinant antibody fragments that preferentially recognized certain metals in complex with iminodiacetic acid have also been reported (Barbas et al., Proc. Natl. Acad. Sci. USA 90:6385-6389 (1993)). Monoclonal antibodies specific for complexes of EDTA-Cd(II), DTPA-Co(II), 2,9-dicarboxyl-1,10-phenanthroline-U(VI), or cyclohexyl-DTPA-Pb(II) have been used in competitive immunoassays for detecting chelated cadmium, lead, cobalt, and uranium (Blake D A, et al. Biosensors & Bioelectronics 16:799-809 (2001)).

Antibodies that specifically bind an analyte can also be made using routine methods. For example, antibodies can be purified from animals immunized with analyte. Monoclonal antibodies can be produced by fusing myeloma cells with the spleen cells from a mouse that has been immunized with the opioid analyte or with lymphocytes that were immunized in vitro. Antibodies can also be produced using recombinant technology.

The capture agent of the disclosed compositions and methods may be an antibody, such as an anti-metatype antibody. Anti-metatype antibodies are immunological reagents specific for the conformation of the liganded antibody active site which do not interact with bound ligand or unliganded antibody. An antibody that selectively binds a capture complex but not to free analyte may be obtained using standard methods known in the art. For example, a naive scFv antibody fragment phage display library may be used to select antibodies that bind to an immunocomplex of analyte and Fab fragments of antibodies that specifically bind the analyte. First the phages are preincubated to sort out those binding to Fab fragments as such. The unbound phages are separated and incubated with a mixture of analyte and immobilized Fab to select the phages that bind to the immunocomplex formed between the immobilized Fab and analyte. Unbound phages are washed away, and then those bound to the complex are eluted. The background is monitored by checking the binding to Fab in the absence of analyte. After several panning rounds a number of clones are picked up, sequenced and expressed resulting in an scFv fragment for use as a capture agent.

ii. Nucleic Acid Aptamers

Nucleic acid aptamers are typically oligonucleotides ranging from 15-50 bases in length that fold into defined secondary and tertiary structures, such as stem-loops or G-quartets. The oligonucleotide may be DNA or RNA and may be modified for stability. A nucleic acid aptamer generally has higher specificity and affinity to a target molecule than an antibody. Nucleic acid aptamers preferably bind the target molecule with a K_{d} less than 10⁻⁶, 10⁻⁸, 10⁻¹⁰, or 10⁻¹². Nucleic acid aptamers can also bind the target molecule with a very high degree of specificity. It is preferred that the nucleic acid aptamers have a K_{d} with the target molecule at least 10, 100, 1000, 10,000, or 100,000-fold lower than the K_{d} with other molecules. In addition, the number of target amino acid residues necessary for aptamer binding may be smaller than that of an antibody.

Nucleic acid aptamers are typically isolated from complex libraries of synthetic oligonucleotides by an iterative process of adsorption, recovery and reamplification. For example, nucleic acid aptamers may be prepared using the SELEX (Systematic Evolution of Ligands by Exponential Enrichment) method. The SELEX method involves selecting an RNA molecule bound to a target molecule from an RNA pool composed of RNA molecules each having random sequence regions and primer-binding regions at both ends thereof, amplifying the recovered RNA molecule via RT-PCR, performing transcription using the obtained cDNA molecule as a template, and using the resultant as an RNA pool for the subsequent procedure. Such procedure is repeated several times to several tens of times to select RNA with a stronger ability to bind to a target molecule. The base sequence lengths of the random sequence region and the primer binding region are not particularly limited. In general, the random sequence region contains about 20 to 80 bases and the primer binding region contains about 15 to 40 bases. Specificity to a target molecule may be enhanced by prospectively mixing molecules similar to the target molecule with RNA pools and using a pool containing RNA molecules that did not bind to the molecule of interest. An RNA molecule that was obtained as a final product by such technique is used as an RNA aptamer. Representative examples of how to make and use aptamers to bind a variety of different target molecules can be found in U.S. Pat. Nos. 5,476,766, 5,503,978, 5,631,146, 5,731,424, 5,780,228, 5,792,613, 5,795,721, 5,846,713, 5,858,660, 5,861,254, 5,864,026, 5,869,641, 5,958,691, 6,001,988, 6,011,020, 6,013,443, 6,020,130, 6,028,186, 6,030,776, and 6,051,698. An aptamer database containing comprehensive sequence information on aptamers and unnatural ribozymes that have been generated by in vitro selection methods is available at aptamer.icmb.utexas.edu.

In a preferred embodiment, a multi-stage SELEX process is used to select aptamers that bind with high specificity and efficiency to an immunocomplex between an antibody and its target analyte or derivatives thereof. The preferred multi-stage SELEX process is required to: (1) differentiate between two related antibodies that have the capacity to bind the analyte or its derivatives; (2) differentiate between an antibody that is bound to the analyte or its derivatives and an antibody that is unbound by the analyte or its derivatives; (3) differentiate between a single antibody that is bound to either the analyte or the analyte's derivatives; and (4) alter of the aptamer's structure upon binding the desired target immunocomplex. The preferred multi-stage SELEX process is conducted in two stages, wherein each stage utilizes a different modified SELEX method. Stage 1 involves enrichment and recombination of the aptamer library using CE-SELEX. Stage 2 involves completing aptamer selection using Structure-switching SELEX. The specific details of this preferred multi-stage SELEX process are demonstrated in Examples 2 and 3.

In some embodiments, selection of nucleic acid aptamers may also include performing non-homologous Random Recombination (NRR). In this step, the DNA library is partially digested into smaller fragments and reassembled into a new library with aptamer sequences containing 80 or more bases per aptamer. The resulting aptamers from the NRR step may vary in length and may possess multiple or shortened binding motifs, which may enhance the likelihood of identifying an optimized nucleic acid aptamer. In some forms, the NRR step of aptamer selection may also include supplementing the PCR reactions with “GC enhancers” to reduce the variance introduced into the aptamer library following CE SELEX and NRR steps. GC enhancers are known in the art and are commercially available for example, from New England Biolabs.

In some embodiments, aptamer selection may include sequencing the aptamers from any one of ten rounds of Structure Switching SELEX using Next-Generation Sequencing (NGS) methods known in the art. In some forms, candidate aptamer sequences may be further evaluated following sequencing for binding efficiency and specificity using Capillary Electrophoresis (CE) and PCR-based binding assays.

In some embodiments, selection of nucleic acid aptamers for an antibody-analyte immunocomplex can include a plurality of rounds of both positive selection and negative selection. Positive selection refers to selecting for aptamers that can bind the immunocomplex, and negative selection refers to selecting for aptamers that do not bind to the antibody or the analyte alone. A combination of positive selection and negative selection can lead to aptamers that are highly specific for the immunocomplex.

In some embodiments, selection of nucleic acid aptamers may also include a reshuffling step. The reshuffling step can be accomplished by dividing the pool of candidate aptamers into two samples. The first sample will be digested using nucleases such as DNase, thereby degrading the full length sequences of the aptamers into smaller units, the size of which is dependent on the amount of nuclease and the incubation time. The digested sample containing aptamer fragments will be mixed with the second sample containing intact aptamers, along with nucleic acid polymerase and nucleic acid ligase. In the mixture, the intact aptamers can hybridize with the aptamer fragments and serve as a template to extend and connect the fragments, thereby resulting in recombination of aptamer sequences. After recombination, primers can be added to the mixture to allow the fragments to be completely restored to full-length sequences. PCR amplification can be performed to generate enough aptamer copies for selections to continue. Preferably, the reshuffling step is performed after one or more rounds of positive and/or negative selection.

In some embodiments, the aptamer is a molecular aptamer beacon. A molecular beacon is a hairpin-shaped oligonucleotide with a fluorophore, and a quencher linked to each end of its stem. The signal transduction mechanism for molecular recognition is based on Förster resonance energy transfer (FRET) and the conformational change of a molecular beacon. The molecular beacon acts like a switch that is normally closed to bring the fluorophore/quencher pair together to turn fluorescence “off”. When binding to a target biomolecule, it undergoes a conformational change that opens the hairpin structure and separates the fluorophore and the quencher, thus turning “on” the fluorescence.

Molecular aptamer beacons were developed to combine the sequence specificity and sensitivity of aptamers with the real-time detection advantages of molecular beacons. Briefly, oligonucleotides containing a nucleic acid aptamer sequence are designed to have complementary DNA or RNA sequences that form a hairpin, which is opened when the aptamer sequence binds its target. Molecular aptamer beacons are described in Cho et al., Annu Rev Anal Chem (Palo Alto Calif.), 2:241-64 (2009), Hamaguchi et al., Anal Biochem., 294(2):126-31 (2001); Li et al., Biochem Biophys Res Commun, 292(1):31-40 (2002).

Typically, the aptamer is a nucleic acid aptamer containing a 10 base pair fixed “structure switching” sequence and a random or variable region having 15 random bases on either side of the fixed “structure switching” sequence. Typically, the structure switching region extends from base 34 to base 45. In some embodiments, the nucleic acid aptamer contains between about 65 base pairs to more than 200 base pairs in length. Preferably, the nucleic acid aptamer contains between about 85 and 150 base pairs in length. More preferably, the nucleic acid aptamer contains between about 75 and 95 bases. In some forms, the nucleic acid aptamer contains one or more binding motifs and/or the ability to structure switch.

Typically, the nucleic acid aptamer is in an unfolded state when bound to the complementary sequence attached to the magnetic bead. The nucleic acid aptamer is released when the immuno-complex or immuno-complexes are added, thereby inducing a structural change within the structure switching region of the nucleic acid aptamer.

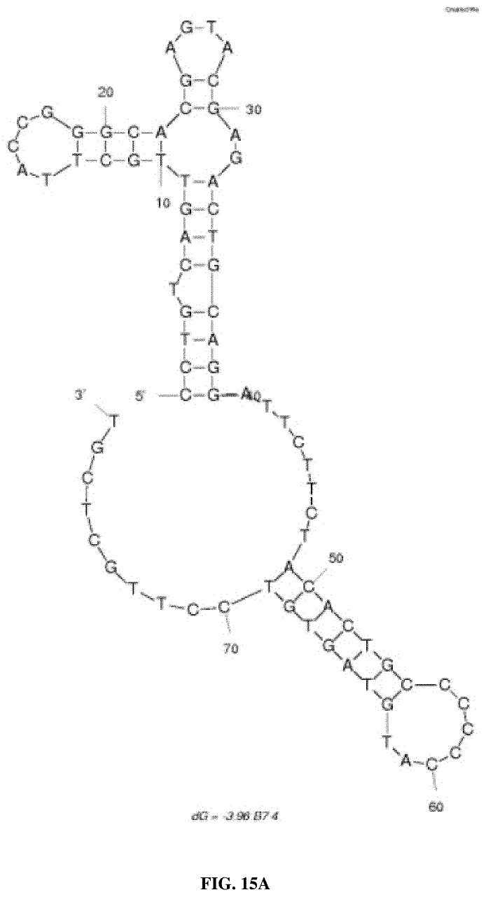

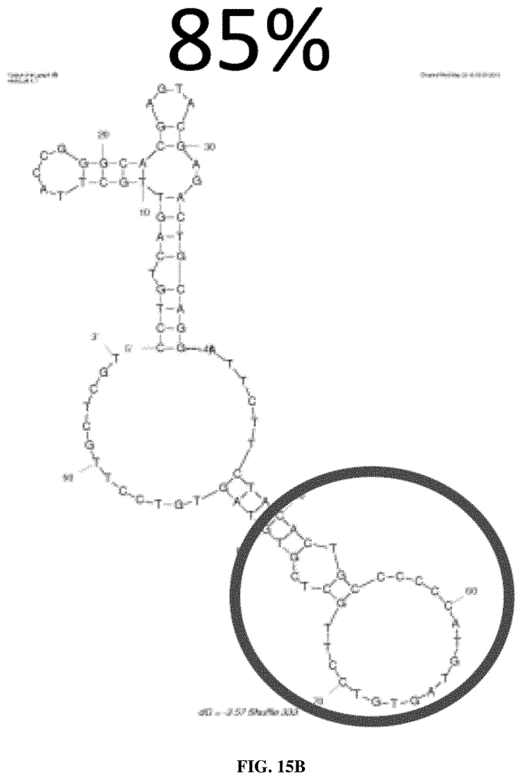

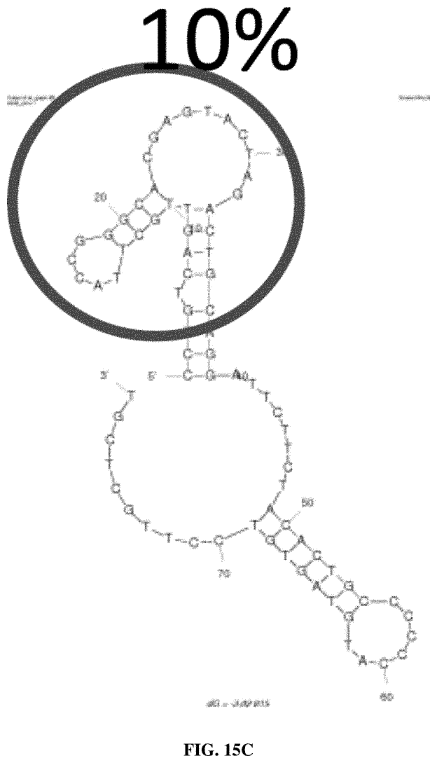

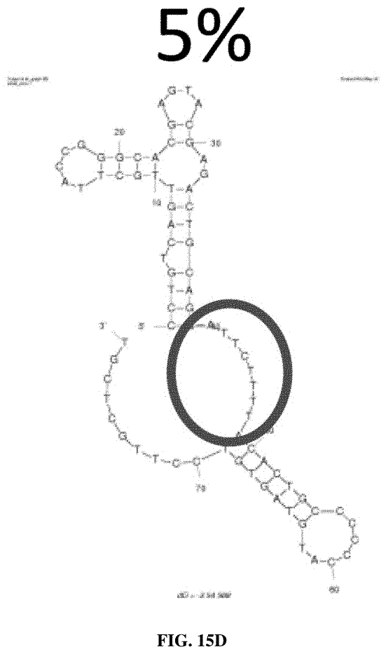

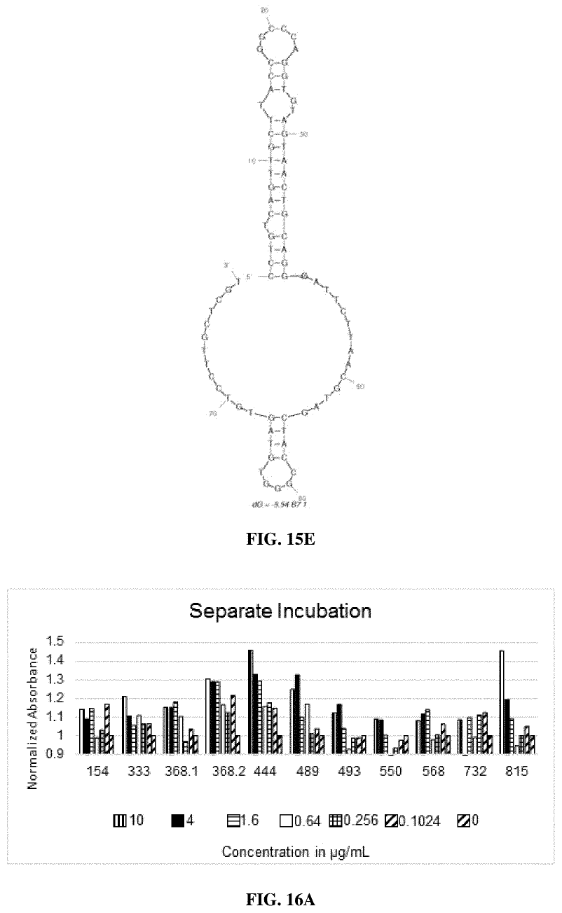

In some embodiments, the nucleic acid aptamer may contain one or more mutations. In some forms, the secondary structures of the nucleic acid aptamer may contain one or more mutations in the fixed structure switching region. In some forms, the nucleic acid aptamer is highly mutated, with ⅔ of the bases in the 3′ random region (associated with structure switching) being different from the parent B7C4-HTHC aptamer. In some forms, the second random regions may be highly mutated. A non-limiting example of a highly mutated aptamer is shown in FIG. 15E. In some forms, the nucleic acid aptamer contains two or more mutations. Preferably, the nucleic acid aptamer contains one or two mutations. More preferably, the nucleic acid aptamer contains a single mutation. In embodiments, the nucleic acid aptamer may contain a mutation in the second random region 3′ to the structure-switching sequence; a non-limiting example is shown in FIG. 15B, SEQ ID NO:34. In these forms, the mutations result in an alteration to the stem loop of the aptamers. In some embodiments, the nucleic acid aptamer may contain mutations in the random region thereby forming base pairs with the structure-switching region; a non-limiting example is shown in FIG. 15C, SEQ ID NO:26. In these forms, the nucleic acid aptamer contains mutations that alter the shape and strength of the structure-switching base pairs; a non-limiting example is shown in FIG. 15C, SEQ ID NO:26. In some embodiments, the nucleic acid aptamers contain mutations in the second random region; a non-limiting example is shown in FIG. 15D, SEQ ID NO:28. In these forms, the nucleic acid aptamers contain mutations in areas that may not have any secondary structure.

In some embodiments, when non-homologous random recombination is included as a step in the selection process, the nucleic acid aptamer may be larger than the aptamer sequences of the parent library. In these forms, the nucleic acid aptamer may contain more than 85 base pairs following one to five rounds of selection of structure switching aptamers (SSAs). In some forms, when non-homologous random recombination is included as a step in the selection process, the nucleic acid aptamer may contain less than 85 base pairs following 10 rounds of selection of structure switching aptamers (SSAs).

In some embodiments, the nucleic acid aptamer may contain one or more modified nucleic acids (also referred to as xeno nucleic acids, or XNAs) for added chemical functionalities that may increase binding affinity of the nucleic acid aptamer to the immuno-complex. Non-limiting modified nucleic acids include but are not limited to unnatural base pairs (UBPs), base modifications such as for example, C7-modified deaza-adenine, C7-modified deaza-gaunosine, C7-modified deaza-cytosine, C7-modified deaza-uridine; and sugar modifications such as for example, ribulonucleic acid, α-L-threose nucleic acid (TNA), 3′-2′ phosphonomethyl-threosyl nucleic acid (tPhoNA) and 2′-deoxyxylonucleic acid (dXNA). In some forms, the modified nucleic acid may be introduced in the nucleic acid aptamer by in vitro evolution using an alternative for the phosphodiester backbone such as for example, phosphorothioates, boranophosphate, phosphonate, alkyl phosphonate nucleic acid, and peptide nucleic acid. In some forms, the modified nucleic acid may be introduced in the nucleic acid aptamer via a mutant T7 RNA polymerase that is tolerant of substitutions at the 2′ position of the furanose ring. Substitutions that may be attached to C2′ include but are not limited to a fluorine, an amine, or a methoxy group. In some forms, the modified nucleic acid may be introduced in the nucleic acid aptamer via R-group modifications at the 5^{th} position of uracil.

In these forms, the R-group can be one of many different sidechains known to those of skill in the art, ranging from hydrophobic to hydrophilic. Incorporation of synthetic nucleotides into nucleic acid aptamers using phosphodiester replacements and modified bases are known to those of skill in the art (See for example, Mayer G. Angew Chem Int Ed Engl. (2009) 48: pages 2672-2689; Keefe, A. D. and Cload, S. T. Curr Opin Chem Biol. (2008); 12: pages 448-456; Appella, D. H. Curr. Opin. Chem. Biol. (2009) 13(5-6): pages 687-696).

In some embodiments, the aptamer is a nucleic acid aptamer having or containing a sequence selected from SEQ ID NOs 1-35.

SEQ ID NO 1:
CCTGTCAGTTGCTTACCGGGCAGGCGAGTAGGACTGCAGCGATTCTTCCT
TGCGGGTGTCGGTGTAGTGTCCTTGCTCGT;
SEQ ID NO 2:
CCTGTCAGTTGCTTACCGGGGCGAGGGACGGAGCTGCAGGATTCTTGCAC
TAGGTGGGGTGTGTAGTGTCCTTGCTCGT
SEQ ID NO 3:
CCTGTCAGTTGCTTACCGGGCGGACGACATAGCCTGCAGCGATTTTCCAA
CACTGCGGTGGAGTAGTGTCCTTGCTCGT
SEQ ID NO 4:
CCTGTCAGTTGCTTACCGGGGACGGCGGTGTGGCTGCAGCGATTCTTAAG
AGGGCCTGGGTGTGTAGTGTCCTTGCTCGT
SEQ ID NO 5:
CCTGTCAGTTGCTTACCGGGGCACGGAGGAACACTGTAGCGATTCTTGTA
TTCGGACCGGTGTGTAGTGTCCTTGCTCGT
SEQ ID NO 6:
CCTGTCAGTTGCTTACCGACACAGACGACATTACTGCAGCCATTCTTCGC
TACGTGCCCGGCTGTAGTGTCCTTGCTCGT
SEQ ID NO 7:
CCTGTCAGTTGCTTACCGGGACGAAGGAAGAAACTGCAGCGATTCTCGTG
GGGCGACGTGTAGTGTCCTTGCTCGT
SEQ ID NO 8:
CCTGTCAGTTGCTTACCGTGTCGCCCAATGAGACTGCAGCGATTCTTTAC
GGATCGGTGTCATGTAGTGTCCTTGCTCGT
SEQ ID NO 9:
CCTGTCAGTTGCTTACCGTGCGGCGAATACGGGCTGCAGCGATTCTTTAG
GGGGTCCACGGGTGTAGTGTCCTTGCTCGT
SEQ ID NO 10:
CCTGTCAGTTGCTTACCGGCACGGTGATCGTACTGCAGGATTCTTCATTT
CGCCGCGTCGTGTAGTGTCCTTGCTCGT
SEQ ID NO 11(B7H357):
CCTGTCAGTTGCTTACCGGGCCGAGCGTACGGACTGCAGCGATTCTTGGT
GACCAGCCGGGGAGTAGTGTCCTTGCTCGT
SEQ ID NO 12(B7HC3):
CCTGTCAGTTGCTTACCGGGCGGAGACGGAAGACTGCAGCAATTCTTGGT
GCTGTGTGTGGCTGTAGTGTCCTTGCTCGT
SEQ ID NO 13(B7HC4):
CCTGTCAGTTGCTTACCGGGCACGAGTACGAGACTGCAGGATTCTTCTAC
ACTGCCCCCCATGTAGTGTCCTTGCTCGT
SEQ ID NO 14(B7HC5):
CCTGTCAGTTGCTTACCGGGCAGCGGTGCAAGACTGCAGGATTCTTCAGA
GGCGGGCCGCGTGTAGTGTCCTTGCTCGT
SEQ ID NO 15(B7H341):
CCTGTCAGTTGCTTACCGGGCACGATGCAAATACTGCAGCGATTCTTGAT
ACCGGTCGCGTGTGTAGTGTCCTTGCTCGT
SEQ ID NO 16(B7H102):
CCTGTCAGTTGCTTACCGACGAAGGCGCTGAAACTGCAGCGATTCTTGCG
GTGTACTGAGTGTGTAGTGTCCTTGCTCGT
SEQ ID NO 17(B7HC1):
CCTGTCAGTTGCTTACCGGCCCAGGTGTAGTAACTGCAGGGATTCTTAAC
GTAGCTACCGGGTGTAGTGTCCTTGCTCGT
SEQ ID NO 18(B7HC2):
CCTGTCAGTTGCTTACCGGGCGGCACATGCTAGCTGCAGCGATTCTTGTA
TTAGGTGCGGCGTGTAGTGTCCTTGCTCGT
SEQ ID NO 19(B7H422):
CCTGTCAGTTGCTTACCGCAGGGCGTCAGGGACCTGCAGCGATTCTTGGT
GTAGGGGGCCGCTGTAGTGTCCTTGCTCGT
SEQ ID NO 20(B7H338):
CCTGTCAGTTGCTTACCGGCACGGGAAGTTGGACTGCAGCGATTCTTTCG
ACGCCCGGCCCCGTGTAGTGTCCTTGCTCGT
SEQ ID NO 21:
CCTGTCAGTTGCTTACCGGGCACGAGTACGAGACTGCAGGATTCTTCTAC
ACTGCTCCCCATGTAGTGTCCTTGCTCGT
SEQ ID NO 22:
CCTGTCAGTTGCTTACCGGCCCAGGTGTAGTAACTGCAGGGATTCTTAAC
GTAGCTACCGGGTGTAGTGTCCTTGCTCGT
SEQ ID NO 23:
CCTGTCAGTTGCTTACCGGGCACGAGTACGAGACTGCAGGATTCTTCTAC
ACTGCGCCCCATGTAGTGTCCTTGCTCGT
SEQ ID NO 24:
CCTGTCAGTTGCTTACCGGGCACGAGTACGAGACTGCAGGATTCTTCTAC
ACTGGCCCCCATGTAGTGTCCTTGCTCGT
SEQ ID NO 25:
CCTGTCAGTTGCTTACCGGGCACGAGTACGAGACTGCAGGATTCTTCTAC
ACTGCACCCCATGTAGTGTCCTTGCTC
SEQ ID NO 26:
CCTGTCAGTTGCTTACCGGGCACGAGTACTAGACTGCAGGATTCTTCTAC
ACTGCCCCCCATGTAGTGTCCTTGCTCGT
SEQ ID NO 27:
CCTGTCAGTTGCTTACCGGCCACGAGTACGAGACTGCAGGATTCTTCTAC
ACTGCCCCCCATGTAGTGTCCTTGCTCGT
SEQ ID NO 28:
CCTGTCAGTTGCTTACCGGGCACGAGTACGAGACTGCAGGATTCTTTTAC
ACTGCCCCCCATGTAGTGTCCTTGCTCGT
SEQ ID NO 29:
CCTGTCAGTTGCTTACCGGGCACGAGTACGAGACTGCAGGATTCTTCTAC
ACTGCCCCACATGTAGTGTCCTTGCTCGT
SEQ ID NO 30:
CCTGTCAGTTGCTTACCGGGCACGAGTACGAGACTGCAGGATTCTTCTAC
ACTGCCCCCCATGTAGTGTCCTTGCTCGTGTAGTGTCCTTGCTCGT
SEQ ID NO: 31:
CCTGTCAGTTGCTTACCCGGGGTGGTGGCTGCGAAGCCGGGATTCTTAAG
AGGGCCTGGGTGTGTAGTGTCCTTGCTCGT
SEQ ID NO: 32
CCTGTCAGTTGCTTACCGTGCGGCGAATACGGGCTGCAGCGATTCTTTAG
GGGGTCCACGGGTGTAGTGTCCTTGCTCGT
SEQ ID NO: 33
CCTGTCAGTTGCTTACCGTGTAGTGTCCTTGCTCGTCCTGTCAGTTGCTT
ACCGTGTAGTGTCCTGCTCGT
SEQ ID NO: 34
CCTGTCAGTTGCTTACCGGGCACGAGTACGAGACTGCAGGATTCTTCTAC
ACTGCCCCCCATGTAGTGTCCTTCCTCGTGTAGTGTCCTTGCTCGT
SEQ ID NO: 35
CCTGTCAGTTGCTTACCGTGTAGTGTCCTTGCTCGTCCTGTTCAGTTGCT
TACCGTGTAGTGTCCTTGCTCGT

In some embodiments, the aptamer is a nucleic acid aptamer having or containing a sequence that has between about 70% and about 100% sequence identity to any one of SEQ ID NOs 1-35. For example, the aptamer may have 70%, 75%, 80%, 85%, 90%, or 95% sequence identity to any one of SEQ ID NOs 1-35.

In some embodiments, the aptamer is a nucleic acid aptamer having or containing a sequence that has between about 70% and 100% sequence identity to the variable sequence region of any one of SEQ ID NOs 1-35.

In some embodiments, the aptamer is a nucleic acid aptamer having or containing a sequence that has between 70% and 100% sequence identity to the structure-switching region of any one of SEQ ID NOs 1-35.

The foregoing aptamers can used for detecting or quantifying cannabinoids such as THC, 11-hydroxy-THC (HTHC), and THC-COOH.

iii. Peptide Aptamers

Peptide aptamers are small peptides with a randomized amino acid sequence that are selected for their ability to bind a target molecule. Peptide aptamer selection can be made using different systems, but the most used is currently the yeast two-hybrid system. Peptide aptamer can also be selected from combinatorial peptide libraries constructed by phage display and other surface display technologies such as mRNA display, ribosome display, bacterial display and yeast display. These experimental procedures are also known as biopannings. Among peptides obtained from biopannings, mimotopes can be considered as a kind of peptide aptamers. All the peptides panned from combinatorial peptide libraries have been stored in a special database with the name MimoDB.

C. Biological Sample

In the disclosed assays, a biological sample is assessed for the presence, absence, or most preferably, the quantity of a small analyte. The biological sample is preferably a bodily fluid, such as whole blood, plasma, serum, urine, cerebrospinal fluid, saliva, oral fluid, semen, vitreous fluid, or synovial fluid. In a preferred embodiment, the bodily fluid is whole blood, plasma, oral fluid, or serum.

Assay Fluid

An aqueous assay fluid can also be introduced to the biological sample, forming a mixed fluid sample. The assay fluid supports a reaction between the analyte and the labeled binding agent (e.g., does not interfere with binding) and has a viscosity that is sufficiently low to allow movement of the assay fluid by capillary action. In some embodiments, the assay fluid contains one or more of the following components: a buffering agent (e.g., phosphate); a salt (e.g., NaCl); a protein stabilizer (e.g., bovine serum albumin “BSA”, casein, serum); and a detergent such as a nonionic detergent or a surfactant (e.g., NINATE® 411, ZONYL® FSN 100, AEROSOL OT 100%, GEROPON® T-77, BIO-TERGE® AS-40, STANDAPOL® ES-1, TETRONIC® 1307, SURFNYOL® 465, SURFYNOL® 485, SURFYNOL® 104PG-50, IGEPAL® CA210, TRITON™ X-45, TRITON™ X-100, TRITON™ X305, SILWET® L7600, RHODASURF® ON-870, CREMOPHOR® EL, TWEEN® 20, TWEEN® 80, BRIJ 35, CHEMAL LA-9, PLURONIC® L64, SURFACTANT 10G, SPAN™ 60). Optionally, if desired, the assay fluid can contain a thickening agent. Representative assay fluids include saline, or 50 mM Tris-HCl, pH 7.2. In some embodiments, the assay fluid is water.

D. Lateral Flow Device

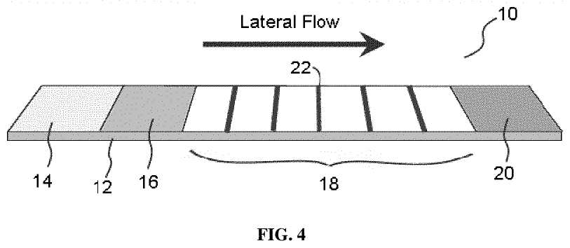

In preferred embodiments, the disclosed point-of-care assay is a lateral flow assay, which is a form of immunoassay in which the test sample flows along a solid substrate via capillary action. As illustrated in FIG. 4, a lateral flow device 10 includes a solid substrate 12, such as a membrane strip, having an application point 14, an optional conjugate zone 16, a capture zone 18, and an absorbent zone 20 (e.g., a wicking pad). Binding agents are optionally present in the conjugate zone 16. Capture agents are immobilized in the capture zone 18, which preferably contains a plurality of capture lines 22 for detecting captured analyte (capture complex).

i. Solid Substrate

The solid substrate 12, such as a membrane strip, can be made of a substance of sufficient porosity to allow movement of antibodies and analyte by capillary action along its surface and through its interior. Examples of suitable membrane substances include: cellulose, cellulose nitrate, cellulose acetate, glass fiber, nylon, polyelectrolyte ion exchange membrane, acrylic copolymer/nylon, and polyethersulfone. In a one embodiment, the membrane strip is made of cellulose nitrate (e.g., a cellulose nitrate membrane with a Mylar backing) or of glass fiber.

In a preferred embodiment, the membrane strip is FUSION 5™ material (Whatman), which is a single layer matrix material that performs all of the functions of a lateral flow strip. For FUSION 5™, the optimal bead size is approximately 2 microns; the FUSION 5™ material has a 98% retention efficiency for beads of approximately 2.5 microns. Beads of 2.5 microns will not generally enter the matrix, whereas beads of below 1.5 microns will be washed out of the matrix.

ii. Application Point

The solid substrate 12 includes an application point 14, which can optionally include an application pad. For example, if the sample containing the analyte contains particles or components that should preferentially be excluded from the immunoassay, an application pad can be used. The application pad typically can filter out particles or components that are larger (e.g., greater than approximately 2 to 5 microns) than the particles used in the disclosed methods.

The application pad may be used to modify the biological sample, e.g., adjust pH, filtering out solid components, separate whole blood constituents, and adsorb out unwanted antibodies. If an application pad is used, it rests on the membrane, immediately adjacent to or covering the application point. The application pad can be made of an absorbent substance which can deliver a fluid sample, when applied to the pad, to the application point on the membrane. Representative substances include cellulose, cellulose nitrate, cellulose acetate, nylon, polyelectrolyte ion exchange membrane, acrylic copolymer/nylon, polyethersulfone, or glass fibers. In one embodiment, the pad is a Hemasep™-V pad (Pall Corporation). In another embodiment, the pad is a Pall™ 133, Pall™ A/D, or glass fiber pad.

iii. Conjugate Zone

The solid substrate 12 optionally contains a conjugate zone 16, which contains binding agents. In some embodiments, the conjugate zone contains binding agents which bind the analyte to be measured and a control analyte. When the sample migrates through the conjugate zone containing binding agents, the analytes in the sample interacts with the binding agents to form capture complexes.

iv. Absorbent Zone

The absorbent zone 20 preferably contains a wicking pad. If a wicking pad is present, it can similarly be made from such absorbent substances as are described for an application pad. In a preferred embodiment, a wicking pad allows continuation of the flow of liquid by capillary action past the capture zones and facilitates the movement of non-bound agents away from the capture zones.

v. Capture Zone

The capture zone 18 contains capture agent immobilized (e.g., coated on and/or permeated through the membrane) to the membrane strip. In preferred embodiments, the capture agent is conjugated to a capture particle that is immobilized in the capture zone 18.

The capture zone 18 is preferably organized into one or more capture lines containing capture agents. In preferred embodiments, the capture zone contains a plurality of capture lines for multiplex analysis, i.e., detection of two or more analytes. In addition, the capture zone 18 may contain one or more control capture lines for detecting the presence of control analyte (i.e., control or calibration capture zone). In preferred embodiments, the control analyte is a compound that is not normally present in any prescription or non-prescription drug, food, beverage, or supplement. Preferably, the control analyte capture reagent specifically binds the control analyte but does not interact with the sample analyte being measured.

The calibration capture zone is preferably positioned such that the sample capture zone is between the application point and the calibration capture zone. In a preferred embodiment, the calibration capture zone is closely adjacent to the sample capture zone, so that the dynamics of the capillary action of the components of the assay are similar (e.g., essentially the same) at both the calibration capture zone and the sample capture zone. For example, the two capture zones are sufficiently close together such that the speed of the liquid flow is similar over both zones. Although they are closely adjacent, the calibration capture zone and the sample capture zone are also sufficiently spaced such that the particles arrested in each zone can be quantitated individually (e.g., without crosstalk). Furthermore, in a preferred embodiment, the sample capture zone is separated from the application point by a space that is a large distance, relative to the small distance between the sample capture zone and the calibration capture zone. Because particle capture is a rate limiting step in the assay, the distance between the application point and the capture zones (where particles are captured) must be sufficient to retard the speed of the liquid flow to a rate that is slow enough to allow capture of particles when the liquid flow moves over the sample capture zone. The optimal distances between the components on the membrane strip can be determined and adjusted using routine experimentation

In some embodiments, the capture zone 18 contains at least one capture line 22 with capture agents for detecting a dilution control analyte, i.e., an analyte that is typically present in the biological sample at predictable concentrations. Creatine is a particularly preferred dilution control analyte when the biological sample is urine. The typical human reference ranges for serum creatinine are 0.5 to 1.0 mg/dL (about 45-90 μmol/L) for women and 0.7 to 1.2 mg/dL (60-110 μmol/L) for men.

In some embodiments, the capture zone 18 contains one or more capture lines with capture agents for detecting reference analytes. The reference analytes may be administered to the biological sample at known concentrations. These reference values can facilitate quantitative correlations between label detection and analyte amounts.

vi. Capture Particles

Capture particles are particles, such as polymeric particles, which can be coated with the capture agent and immobilized to the membrane in the capture zone 18. In preferred embodiments, the particles are physically trapped within the membrane. This allows for selection of optimal particle chemistry that is not influenced by the need for chemical immobilization. Suitable capture particles include liposomes, colloidal gold, organic polymer latex particles, inorganic fluorescent particles, and phosphorescent particles. In some embodiments, the particles are polystyrene latex beads, and most particularly, polystyrene latex beads that have been prepared in the absence of surfactant, such as surfactant-free Superactive Uniform Aldehyde/Sulfate Latexes (Interfacial Dynamics Corp., Portland, Oreg.).

In preferred embodiments, the particles are monodispersed polymer microspheres based on melamine resin (MF) (e.g., available from Sigma-Aldrich). Melamine resin microspheres are manufactured by acid-catalyzed hydrothermal polycondensation of methylol melamine in the temperature range of 70-100° C. without any surfactants. Unmodified MF particles have a hydrophilic, charged surface due to the high density of polar triazine-amino and -imino groups. The surface functional groups (methylol groups, amino groups, etc.) allow covalent attachment of other ligands. For special applications, the MF particles can be modified by incorporation of other functionalities such as carboxyl groups. This increases possible surface derivatization such as chromophore or fluorophore labeling.

The particles can be labeled to facilitate detection by a means which does not significantly affect the physical properties of the particles. For example, the particles can be labeled internally (that is, the label is included within the particle, such as within the liposome or inside the polystyrene latex bead). Representative labels include luminescent labels; chemiluminescent labels; phosphorescent labels; fluorescent labels; phosphorescent labels; enzyme-linked labels; chemical labels, such as electroactive agents (e.g., ferrocyanide); and colorimetric labels, such as dyes. In one embodiment, a fluorescent label is used. In another embodiment, phosphorescent particles are used, particularly up-converting phosphorescent particles, such as those described in U.S. Pat. No. 5,043,265.

The particles are preferably coated with capture agent, such as a sample analyte capture agent and control analyte capture agent. They can be prepared by mixing the capture agent in a conjugation buffer. A covalent coupling onto the particles is then performed, resulting in random binding of the capture agents onto the particle.

E. Sample Collection Apparatus

The quantitative point-of-care assay may involve the use of a sample collection apparatus that is not in fluid contact with the solid phase apparatus. The sample collection apparatus can be any apparatus which can contain binding agents and to which a measured volume of fluid sample can be added. Representative sample collection apparatus includes a sample tube, a test tube, a vial, a pipette or pipette tip, or a syringe. In a preferred embodiment, the sample collection apparatus is a pipette or pipette tip.

In one embodiment, the sample collection apparatus contains a population of binding agents. The binding agents can be stored within the sample collection apparatus in a stable form, i.e., a form in which the agents do not significantly change in chemical makeup or physical state during storage. The stable form can be a liquid, gel, or solid form. In preferred embodiments, the agents are evaporatively dried; freeze-dried; and/or vacuum dried. In one preferred embodiment, the sample collection apparatus contains a pipette tip having vacuum-dried binding particles within its tip. In another preferred embodiment, the sample collection apparatus contains a pipette tip having vacuum-dried analyte binding particles and vacuum-dried calibration analyte binding particles within its tip.

In other embodiments, the sample collection apparatus contains a population of drug binding particles and a population of calibration analyte binding particles. The sample collection apparatus may also contain calibration analyte. If so, the population of particles is located at a different place in the sample collection apparatus from the calibration analyte. The calibration analyte can also be evaporatively dried, vacuum-dried or freeze-dried in the sample collection apparatus. If the calibration analyte is not stored within the sample collection apparatus, then it can be present in the assay fluid.

In either embodiment, the population of particles varies, depending on the size and composition of the particles, the composition of the membrane of the solid phase apparatus, and the level of sensitivity of the assay. The population typically ranges approximately between 1×10³ and 1×10⁹, although fewer or more can be used if desired. In certain embodiments, the amount of particles is determined as an amount of solids in the suspension used to apply the particles for storage within the sample collection apparatus. For example, when applying the particles in solution for freeze- or vacuum-drying in the sample collection apparatus, a suspension of approximately 0.05% to 0.228% solids (w/v) in 5 μl of suspension can be used. Alternatively, other amounts can be used, including, for example, from approximately 0.01% to 0.5% (w/v).

The binding particles (coated with both drug binding agent and calibration analyte binding agent), or the analyte binding particles and the calibration analyte binding particles, can be stored within the sample collection apparatus in a stable form, i.e., a form in which the particles do not significantly change in chemical makeup or physical state during storage. The analyte binding particles and the calibration analyte binding particles are stored at the same location within the sample collection apparatus (e.g., applied as a homogeneous mixture to the location).

F. Magnetic Beads/Particles

In some embodiments, the point-of-care assay described herein uses magnetic beads/particles, which are conjugated with the binding agent or the capture agent. Preferably, the magnetic beads/particles are conjugated with the capture agent, such as aptamers, which can detect immunocomplexes formed of analytes and their antibodies in solution. Preferably, the detectable label is linked to the free agent in solution, i.e., the agent not conjugated to the magnetic beads/particles. For example, if the capture agent is conjugated with the magnetic beads/particles, the binding agent is the free agent and is thus labeled with the detectable label, and vice versa. When there are multiple types of free agents in solution, e.g., multiple types of binding agents, the free agents can be labeled with a single type of detectable label for total analyte detection or labeled with different types of detectable labels for simultaneous detection of a group of analytes.

Conventional lateral flow immunoassays usually have up to 12 detection lanes, whereas the use of magnetic beads/particles can allow for simultaneous detection of a larger number of analytes. In some embodiments, this can be achieved by using either (a) magnetic beads/particles conjugated with different types of capture agents (e.g., aptamers), and/or (b) a mixture of different groups of magnetic beads/particles, wherein each group is conjugated with a distinct type of capture agent.

The use of magnetic beads/particles is advantageous for detection of a class of compounds, such as opioids or cannabinoids. When simultaneously detecting a group of structurally similar compounds, such as THC, 11-hydroxy-THC, and tetrahydrocannabinolic acid, the magnetic bead-based assays may simply require the use of a single antibody, which has septicity for all of the structurally similar compounds. The magnetic beads/particles can be conjugated with different types of aptamers, with each type of aptamers recognizing a distinct immunocomplex formed of a specific compound and its antibody.

The binding agent or capture agent can be conjugated to the magnetic beads/particles through covalent or noncovalent interactions, preferably through covalent interactions. The magnetic beads/particles can have an average size in the range of 500 nm to 200 m. The magnetic beads/particles contain a magnetic core that are typically ferrimagnetic or superparamagnetic. The magnetic core can be covered by a range of different materials, providing different properties. For example, agarose forms a three-dimensional hydrophilic mesh out of linear sugar polymers with neutral charge. The polymer is chemically crosslinked to provide thermal and mechanical stability (e.g., SEPHAROSE®). The large interacting surface leads to high binding capacities. The neutral surface reduces non-specific binding. Silica-based beads/particles are widely used for nucleic acid purification. They become negatively charged at pH>3.

The surface of the magnetic beads/particles may have a plurality of one or more of the following chemical groups, through which the magnetic beads/particles are conjugated with the binding agent or the capture agent: aldehyde (e.g., for coupling to amine groups), carboxyl (e.g., for coupling to amine groups), hydrazide (e.g., for coupling to aldehyde groups), streptavidin (e.g., for coupling to biotinylated agents), biotin (e.g., for coupling to streptavidin-labeled agents), sulfonic group (e.g., for coupling to amine groups), epoxy (e.g., for coupling to amine groups), isothiocyanate (e.g., for coupling to amine groups), tosyl (e.g., for coupling to amine groups), hydroxyl (e.g., for coupling to silane groups), amine (e.g., for coupling to carboxyl groups), N-hydroxysuccinimide (e.g., for coupling to amine groups), and maleimide (e.g., for coupling with thiol groups). The binding agent or capture agent may endogenously contain the chemical entity capable of coupling to the chemical groups on the surface of the magnetic beads/particles. Alternatively, the binding agent or capture agent can be functionalized with such a chemical entity so that it can be conjugated to the surface of the magnetic beads/particles. For example, nucleic acid aptamers can be labeled with an amine group at either the 3′ end or the 5′ end. Exemplary coupling methods can be found in Goda et al., Current Physical Chemistry, 2011, 1(4):276-291 and Rashid et al., Sensing and Bio-Sensing Research, 2017, 16.

The easy and efficient collection of magnetic beads/particles in magnetic fields allows for easy rinsing and removal of excess reagents. This approach does not require columns or centrifugation steps and are therefore ideal in high-throughput and automated applications.

A general workflow for detect or quantitatively measuring the amount of an analyte in a biological sample from a subject can include: (1) contacting magnetic beads/particles, coupled with the binding agent or the capture agent, to the biological sample, in the presence of the capture agent or the binding agent, respectively, (2) incubating the resulting mixture under conditions to allow formation of the sandwich complex of the analyte, the binding agent, and the capture agent; (3) capture the magnetic beads/particles with magnet; (4) wash the magnetic beads/particles, and (5) detect or quantify the amount of the sandwich complex. The assay can be performed in an assay fluid as described above.

In the presence of a magnet, the magnetic beads/particles respond to the magnetic field, allowing bound material to be rapidly and efficiently separated from the rest of the sample. Unbound material can be simply removed by aspiration, and the bound material can be further washed using the magnet.

In some embodiment, the bound material can be released in a suitable volume for use in downstream detection or quantification. Alternatively, the bound material can be detected or quantified directly while still attached to the beads. The sandwich complex isolated by the magnetic beads/particles can be detected or quantified based on the detectable label(s) attached to the binding agent and/or capture agent.

### III. Assay Methods

The lateral flow assay can be used to detect a small analyte, such as drug, drug metabolite, heavy metal, or hormone, in a biological sample. The assay generally involves combining the biological sample with an assay fluid, a drug binding agent that specifically binds a drug analyte, a calibration/control analyte, and a calibration/control binding agent that specifically binds the calibration analyte. Contacted capture particles may or may not have analyte bound to the analyte binding agent, depending on whether analyte is present in the fluid sample and whether analyte has bound to the analyte binding agent on the binding particles. Because there are multiple binding sites for analyte on the capture particles, the presence and the concentration of analyte bound to particles varies; the concentration of analyte bound to the particles increases proportionally with the amount of analyte present in the fluid sample, and the probability of a particle being arrested in the sample capture zone similarly increases with increasing amount of analyte bound to the drug binding agent on the particles. Thus, the population of contacted binding particles may contain particles having various amount of analyte bound to the drug binding agent, as well as particles having no analyte bound to the drug binding agent. In some preferred embodiments, only the mobile element contains a label.

In a preferred embodiment, the drug analyte and the control analyte have similar physical properties. For example, the control analyte is preferably a small molecule of similar size to the drug analyte of interest. However, the calibration analyte is preferably not present in human biological samples and does not cross-react with the drug binding agent. Therefore, in preferred embodiments, the calibration analyte is a compound that is not normally present in any prescription or non-prescription drug, food, beverage, or supplement.

In another preferred embodiment, the drug binding agent and the control binding agent also have similar properties. For example, if the drug binding agent is an antibody, the calibration binding agent is also preferably an antibody.

Moreover, the affinity and/or avidity of the calibration/control binding agent for the calibration/control analyte is preferably comparable (e.g., within one order of magnitude) to the affinity and/or avidity of the drug binding agent for the drug analyte.

A. Sample Preparation

In one embodiment, the biological sample is first combined with a binding agent in an assay fluid to produce a mixed fluid sample. If analyte is present in the mixed fluid sample, binding occurs between the analyte and the binding agent to produce capture complex. The degree of binding increases as the time factor of the conditions increases. While the majority of binding occurs within one-minute, additional incubation for more than one minute, 2 minutes, 5 minutes, 10 minutes, or 15 minutes results in additional binding. In some embodiments, the binding agent is present in the sample collection apparatus. The biological sample is preferably mixed with calibration analyte and particles coated with a calibration binding agent. In preferred embodiments, the binding particles contain detectable labels.

If there is no calibration analyte in the sample collection apparatus, then the assay fluid can contain calibration analyte. Therefore, the mixed fluid sample contains drug binding particles, calibration binding particles, calibration analyte and sample analytes (if present).

In still other embodiments, the binding agents are present in the conjugation zone of the lateral flow membrane strip. In these embodiments, the sample is collected into any sample collection container used in the art to collect such samples, for example, any common laboratory container for collecting random urine samples can be used to collect urine. Samples should be collected following recommended guideline known in the art to avoid false negative results as described with respect to urine samples for example in Moeller et al., Mayo Clin. Proc., 83(1):66-76 (2008).

B. Application of Sample

The sample is applied to the application point 14 of the membrane strip, or to the application pad, if present. After the membrane strip is contacted with the sample, the membrane strip is maintained under conditions (e.g., sufficient time and fluid volume) which allow the labeled binding agents to move by capillary action along the membrane to and through the capture zone 18 and subsequently beyond the capture zones 18 (e.g., into a wicking pad), thereby removing any non-bound labeled binding agents from the capture zones. In some embodiments, the sample migrates through the conjugate zone containing binding agents. The analyte in the sample interacts with the binding agents to form capture complexes.

As the applied sample passed through the membrane strip, analyte bound (sample/control analyte) to binding agent (capture complex) are immobilized by capture agents in the capture zone 18, which are preferably conjugated to immobilized capture particles. The capture zone 18 is preferably organized into one or more capture lines in specific areas of the capture zone where they serve to capture the capture complexes as they migrate by the capture lines. The capture zone 18 preferably contains a plurality of capture lines 22 for multiplex analysis and quantification.

Capillary action subsequently moves any binding agents that have not been arrested onwards beyond the capture zone 18, for example, into a wicking pad which follows the capture 18 zone. If desired, a secondary wash step can be used. Assay fluid can be applied at the application point after the mixed fluid sample has soaked into the membrane or into the application pad, if present. The secondary wash step can be used at any time thereafter, provided that it does not dilute the mixed fluid sample. A secondary wash step can contribute to reduction of background signal when the capture particles are detected.

C. Detection

The amount of analyte bound by binding agents arrested in the capture zone (sandwich complex) may then be detected. The labeled binding or capture agents are preferably detected using an appropriate means for the type of label used. In a preferred embodiment, the labeled binding or capture agents are detected by an optical method, such as by measuring absorbance or fluorescence. In preferred embodiments, the particles are detected using an ESEQuant™ Lateral Flow Immunoassay Reader (Qiagen). Alternatively, labeled binding or capture agents can be detected using electrical conductivity or dielectric (capacitance). Alternatively, electrochemical detection of released electroactive agents, such as indium, bismuth, gallium, or tellurium ions, or ferrocyanide can be used. For example, if liposomes are used, ferrocyanide encapsulated within the liposome can be released by addition of a drop of detergent at the capture zone, and the released ferrocyanide detected electrochemically. If chelating agent-protein conjugates are used to chelate metal ions, addition of a drop of acid at the capture zone will release the ions and allow quantitation by anodic stripping voltammetry. Alternatively, magnetic particle detection methods as well as colorimetric methods can be utilized.

D. Interpreting Results

For non-competitive assays, the amount of analyte in the sample is directly related to the level of detection agent detected in a capture line. This value is preferably normalized by the amount of another detectable label immobilized within the membrane (e.g., capture zone) to account for variations in detection device and parameters (e.g., light intensity). This normalized value may then be plotted against a standard curve or response surface that correlates these normalized values to analyte concentration. For example, a standard curve or response surface may be prepared in advance using analyte standards. In addition, three or more internal standard analytes may be detected in the assay and used to adjust or select the standard curve or surface from reference curves or surfaces.

The response surface methodology (RSM) is a collection of mathematical and statistical techniques useful for the modeling and analysis of problems in which a response of interest is influenced by several variables and the objective is to optimize this response (Montgomery, Douglas C. 2005. Design and Analysis of Experiments: Response surface method and designs. New Jersey: John Wiley and Sons, Inc.). In some cases, a fitted RSM model is used to determine the analyte concentration more accurately from a multiplexed assay with a range of detection agents. For example, the binding of analyte to the capture agent is dependent both on the specific agent x₁ (e.g., antibody) and the concentration of the analyte x₂ (e.g., THC). The test can be conducted with combinations of x₁ (continuous variable) and x₂ (cardinal variable) to determine a response to analyte values (continuous variable). The cardinal value can constitute the physical ordering on the test strip (e.g., line 1, line 2, etc.).

However, as the RSM is fitted to minimize error and not intrinsically related to the actual physical ordering of the binding agents in the assay, other orderings (i.e., ordinal, continuous) may be preferred to simplify the fit. In the simplest case, the fluorescent intensity y is the response variable, and this detected intensity is a function of analyte concentration (x₁) and the binding agent used (x₂). This function can be expressed as

y=ƒ(x₁,x₂)+ε.

The variables x₁ and x₂ are independent variables where the response y depends on them. Additional independent variables (e.g., x₃, x₄, etc.) may also be used to improve quantitative results. The dependent variable y is a function of x₁, x₂, and the experimental error term, denoted as E. The error term ε represents any measurement error on the response, as well as other type of variations not counted in ƒ. This is a statistical error that is assumed to be distributed normally with zero mean and variance s². In most RSM problems, the true response function ƒ is unknown. To develop a proper approximation for ƒ, the experimenter starts with a low-order polynomial in a small test region. If the response can be defined by a linear function of independent variables, then the approximating function is a first-order model. A first-order model with two independent variables can be expressed as

y=β₀+β₁x₁+β₂x₂+ε.

If there is a curvature in the response surface, as is commonly the case with binding curves, then a higher degree polynomial should be used. The approximating function with two variables is called a second-order model:

y=β₀+β₁x₁+β₂x₂+β₁₁x²₁₁+β₂₂x²₂₂+β₁₂x₁x₂+ε

Higher order models are possible but in general all RSM problems use either one or a mixture of both models.

Given the RSM equation where y is the detected signal and the position of the signal is known to be associated with a particular binding agent x₂, then one can solve for the unknown concentration x₁, where x₁ is a positive real value.

In the preferred embodiment, the response value y is the normalized intensity. This normalization removes noise associated with variations in light intensity resulting from the light source (i.e., aging, warm up, low frequency drift, etc.). Fluorescence detection that is dependent upon analyte concentration (e.g., on binding agents or aptamer beacons) is preferably normalized against another fluorescence marker present in or on the membrane. For example, a fluorescent bead, optionally at the same excitation and emission wavelengths as the fluorescent label dependent upon analyte concentration, may be included in a separate control line to normalize the detection output. This can be represented by the formula

yₙ=yₓ/y_{c},

where yₓ is the detected response of the unknown analyte, y_{c} is the detected response of the control marker in or on the membrane, and yₙ is the normalized response.

In the preferred embodiment, the analyte concentration value x₁ is a dimensionless value scaled by the highest detection concentration for specific analyte associated with the respective binding agent. This simplifies the RSM fitting by putting the various detection analytes on similar scales although the diagnostically relevant detection ranges may be vastly different between the respective analytes (i.e., fentanyl vs. morphine). This can be represented by the formula

xₗₙ=x₁/x_{C},

where xₗₙ is the dimensionless concentration of the unknown analyte, x₁ is the concentration of the analyte, and x_{c} is the concentration of the highest level of analyte in the assay (i.e., higher levels not diagnostically relevant). To recover the actual value of analyte from the dimensionless value derived from the RSM, one just multiplies by the constant x_{c} for that analyte. Typically, this operation would be internal to the device operation and invisible to the end user that would just see a reported concentration for the detected analyte.

In the preferred embodiment, the binding agent x₂ is expressed as a continuous value by ordering the determined calibration curves for each analyte and binding agent and determining a value x₂ for each binding to give the simplest RSM with minimal error. The naive case would order these in a cardinal manner such that the lowest response curves were first and progressed to steeper responses. However, as the physical location of the detection lines are not related to the ordering for the determined surface, continuous values can be assigned to optimize the RSM fitting (i.e., morphine=0.2, fetanyl=1.1, etc. . . . )

In the preferred embodiment, this optimized RSM surface determined by testing known combinations of analytes and binding agents is used to solve for unknown analyte concentrations. Inclusion of internal standards improves this calculation by ensuring that for a given test the determined values are within expected variances (e) or if not can be adjusted to compensate and improve the accuracy of the individual test. In the simplest example, the included internal standards may indicate an error associated with a constant offset, β₀+β′, and can correct the results by subtracting the determined offset, β′, from the formulae to determine unknown values. Inclusion of three or more standards would allow more complex corrections to the RSM surface, including curvature corrections, without having to derive an entirely new model. Preferably five internal standards would be used and cover the four extremities of the RSM model plus a center point.

### IV. Kits

Kits for use in the disclosed methods are also provided. In one embodiment, the kit includes the lateral flow device disclosed herein, which optionally includes a conjugate zone 16, which preferably comprises a binding agent. The kit optionally contains a sample collection apparatus.

In some embodiments, the sample collection apparatus which is not in fluid contains the lateral flow device. In some embodiments, the sample collection apparatus contains a population of binding agents which are preferably, evaporatively, freeze- or vacuum-dried onto the sample collection apparatus. Kit components additionally can include analytes at known concentrations for generating a standard curve, capture particles, particles and conjugation buffer for coating particles with binding agents, disposal apparatus (e.g., biohazard waste bags), and/or other information or instructions regarding the sample collection apparatus (e.g., lot information, expiration date, etc.).

In some embodiments, kits for use in the disclosed methods include magnetic beads/particles. The magnetic beads/particles can be pre-conjugated with the binding agent or the capture agent. Alternatively, the magnetic beads/particles are not pre-conjugated with the binding agent or the capture agent, and will be conjugated with the binding agent or the capture agent by the user. The magnetic beads/particles in the kits may be in the form of a liquid suspension or dry powder.

### EXAMPLES

### Example 1: Aptamers Selection

The antibodies shown in Table 2 are useful in a proof-of-concept assay to identify aptamers that can be used in non-competitive assay for oxycodone. Hydromorphone can be used as a negative control. The antibodies all have cross reactivity to oxycodone, hydrocodone, oxymorphone, noroxycodone, and hydromorphone as shown in Table 2. The structures of oxycodone, hydrocodone, oxymorphone, noroxycodone, and hydromorphone are shown below.

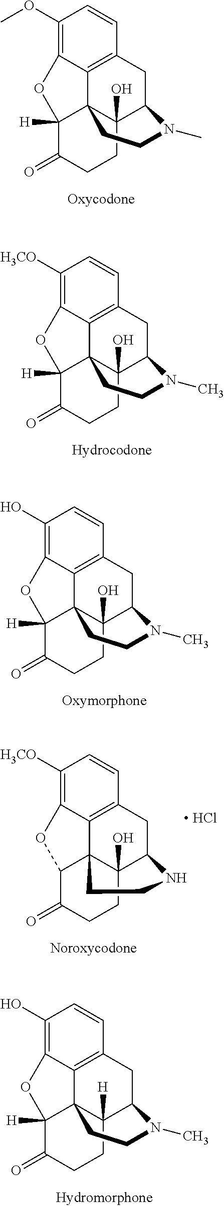

The antibodies used for proof of concept, and relative activity to oxycodone are shown in Table 2.

TABLE 2
Antibodies and their relative activity to oxycodone

| | Relative | | Relative | | |
|---|---|---|---|---|---|
| | Activity | | Activity | | |
| | to | | to | | |
| Antibody/ | Oxycodone | Hydrocodone | Oxymorphone | Noroxycodone | Hydromorphone |
| PAS9713 | 100 | 4.1 | 13.2 | 0.1 | 0.2 |
| PAS9771 | 100 | 2282.4 | 4.4 | 0.1 | 163 |
| PAS9712 | 100 | 34.3 | 0.1 | 19.3 | 0.1 |
| *MBS315355 | 100 | 3.7 | 47.2 | ND | 0.7 |

*Source of antibody is rabbit. All other antibodies are raised in sheep

PAS9713 and PAS9712 are sheep polyclonal antibodies with oxycodone as their target, available from Randox Life Sciences. PAS9771 is an anti-hydromorphone antibody available from Randox Life Sciences. MBS315355 is an anti-oxycodone antibody raised in rabbit, available from MyBioSource, San Diego, Calif. Although PAS9713, PAS9712, and PAS9771 are anti-oxycodone antibodies, they cross react with hydrocodone, oxymorphone, and hyromorphone as shown in Table 2.

Aptamers selective for a drug of interest, for example, oxycodone, can be selected using the in vitro process, SELEX. Aptamers are selected based on their recognition of an oxycodone immunocomplex.

Briefly, the SELEX process begins with a large random oligonucleotide library (pool), whose complexity and diversity are dependent on the number of its random nucleotide positions. Mayer, Anew. Chem. Int. Ed., 48:2672-2689 (2009). During the SELEX procedure, binding DNA from the sequences are separated from DNA lacking affinity. This can be accomplished by immobilizing the target of interest, for example, the antibody-drug complex, to a column matrix, usually agarose or sepharose, and allowing easy partitioning of unwanted sequences through multiple washes. Alternatively, magnetic beads can be used as the solid matrix, as described for the FluMag SELEX system, Stoltenburg, et al., Anal. Bioanal. Chem. 383:83-91 (2005). This results in an enriched pool, which is subjected to further selection rounds that serve to increase the pool's affinity for the target molecule (positive selections) or eliminate members of the pool that have affinity for undesirable compounds (negative selections). After several rounds, the enriched pool is cloned, sequenced, and characterized to find aptamers which show selectivity for the drug of interest. Aptamer binding to antibody (without drug) can be used in a negative selection assay, to select out aptamers which show non-specific binding to antibodies. Negative selection for non-specific binding to immunocomplex (using hydromorphone for example) can also be to enrich the aptamer pool selective to oxycodone.

### Example 2: Generation and Evaluation of Aptamers for Antibody and Analyte Immunocomplexes of THC and its Derivatives

Materials and Methods

Experiments were performed in which aptamers were developed and tested against antibody-small molecule complexes were selected to enable sandwich type assays, not feasible with currently existing antibody technology. Assays for the detection and/or quantification of small molecules typically employ a competitive method, whereby a labeled analyte is bound by an antibody. The labeled analyte is then competed off with a sample containing an unknown amount of unlabeled analyte, resulting in a concentration-dependent decrease in the signal from the labeled analyte. While such competitive assays can allow detection of small molecules using antibodies, they are generally less sensitive and less specific than sandwich type assays. Traditional two-antibody assays in which both antibodies recognize an unmodified analyte are not possible for small molecule detection because the first antibody generally blocks access of the second antibody to the small molecule.

In this non-limiting example, nucleic acid aptamers were developed to replace the second antibody in a sandwich type assay for small analytes, such as tetrahydrocannabinol (THC) and its derivatives. This approach is practical because aptamers generally are smaller and have higher specificity compared to antibodies. Also, aptamers can bind to 3D conformations whereas antibodies often recognize linear strings of peptides. The current assay was designed to facilitate aptamer recognition of the immunocomplex between the antibody and the analyte, but not the antibody or analyte alone.

Selecting an aptamer against a small molecule/antibody immunocomplex has not been previously reported. A multi-stage SELEX (Systematic Evolution of Ligands by Exponential Enrichment) process was developed to select aptamers for an immunocomplex between an antibody and either Tetrahydrocannabinol (THC) or its metabolite, 11-hydroxy-THC (HTHC). A typical SELEX process involves (1) incubating a library of aptamers with random oligonucleotides with a target, (2) removing unbound non-specific aptamers via washing during multiple rounds of selection; (3) enriching the specific aptamers from the pool (4) characterizing the specific aptamers via multiple bioanalytical and biological assays; and (5) either (a) further developing the successful specific aptamers for applications such therapeutics and diagnostic applications or (b) feeding the unsuccessful specific aptamers back into the SELEX cycle.

The desired multi-stage SELEX process was required to: (1) differentiate between two related antibodies that have the capacity to bind either THC or HTHC; (2) differentiate between an antibody that is bound to THC or HTHC and an antibody that is unbound by a THC or HTHC; (3) differentiate between a single antibody that is bound to either THC or HTHC; and (4) alter of the aptamer's structure upon binding the appropriate target. In the current experiment, the multi-stage SELEX process entailed: (1) enrichment and recombination of the aptamer library in Stage one using CE-SELEX, and (2) selection completion of the aptamers using Structure-switching SELEX.

More detailed methods are described as follows.

Antibody Affinity Tests

Two antibodies, THC.5B7 and THC2.B9, with specificity for THC were obtained from Bioventix. The affinities of these antibodies for specific THC metabolites were unavailable. Horseradish Peroxidase (HRP) conjugates of each metabolite (from United Immunoassay) were used to assess the specificity of each antibody for THC or its metabolite, HTHC. The THC.5B7 and THC2.B9 antibodies were coated onto ELISA plates and incubated with a high concentration of either THC-HRP or HTHC-HRP to saturate them with either metabolite. Varying concentrations of the unmodified metabolites were added to each well and incubated. Following a 1-hour incubation period, the plates were washed, and the displacement of the conjugated metabolites was assessed using a 3,3′,5,5′-Tetramethylbenzidine (TMB) colorimetric assay. Less reagent conversion indicates the displacement of the conjugate by the metabolite, and less overall higher affinity of the antibody for the metabolite. The HTHC and THC binding affinity was expressed as a HTHC/THC ratio. Values greater than 1 indicated that THC is preferred over HTHC, and values less than 1 indicated that HTHC is preferred.

Selection of Aptamers Using CE-SELEX

The first stage of the SELEX process included four rounds of positive selection and one round of negative selection. Capillary Electrophoresis (CE) was chosen above other SELEX methods in these initial rounds of aptamer selection for the following reasons: (1) it was previously shown to require fewer rounds of library enrichment; in some cases, as few as 3 rounds were needed to develop specific aptamers; and (2) CE SELEX has the advantage of selecting aptamers that recognize their targets in free solution, as opposed to other methods which require either the aptamer library or target to be bound to a solid support. An enriched library of binding sequences was created using CE SELEX prior to selection with Structure Switching SELEX. Enrichment prior to the selection of Structure Switching Aptamers (SSA's) is beneficial due to the relatively low ability of Structure Switching SELEX to partition binding and non-binding sequences.

The aptamer library was designed with (i) fixed primer sequences on the 5′ and 3′ ends of the aptamer sequences, and (ii) 15 random bases on either side of a 10-base fixed sequence to be used in the Structure Switching SELEX Stage.

The first round of SELEX entailed positive selection, performed by first folding 1 μM of the aptamer library at 80° C. for 15 minutes in 50 mM Tris (pH 8.2), then immediately placing the tube on ice for 15 minutes. 25 μL of the folded library was mixed with 0.2 μL of either B7-HTHC (1:100 ratio) or B9-THC (1:100 ratio) for 30 minutes prior to running the selection. 31 mM Tris and 500 mM Glycine (pH 8.2) was used as the running buffers for the selection. All the DNA that eluted from the column until 30 seconds prior to the “bulk DNA peak” was collected and subjected to PCR analysis using a phosphorylated primer as the basis for the “antisense” primer. The resulting (double stranded) dsDNA from the PCR was then degraded to (single stranded) ssDNA using lambda exonuclease. Lamda exonuclease selectively degrades 5′ phosphorylated dsDNA into ssDNA by degrading the phosphorylated strand. The ssDNA was then purified using a nucleotide removal kit (Qiagen). This process was repeated 3 rounds for positive selection.

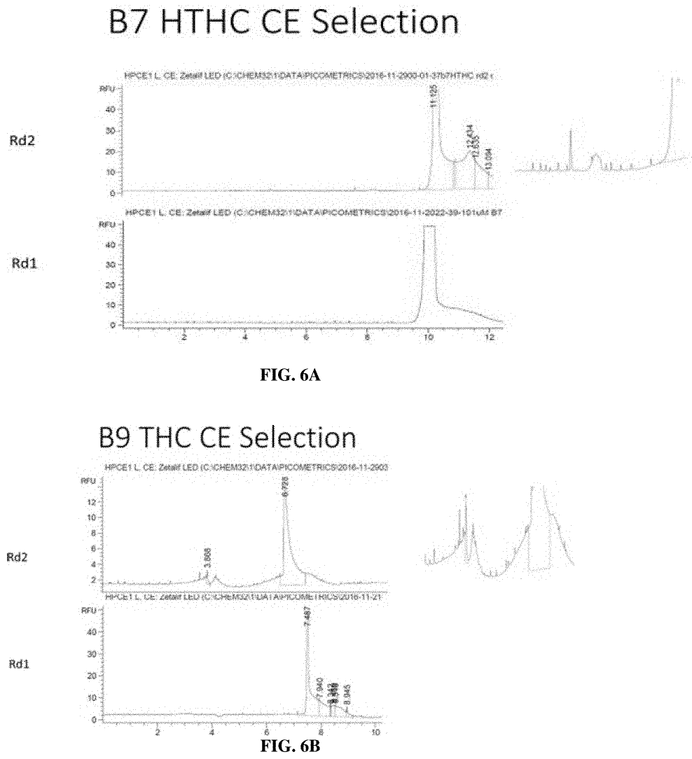

The next round of SELEX was the negative selection of the non-specific aptamers. For negative selection, the aptamers were run in a similar manner as described for positive selection, but no metabolite was added to the antibodies prior to mixing with the aptamers. The bulk DNA peaks were collected for PCR and purification. FIGS. 6A and 6B are representative electropherograms of aptamer samples generated via CE SELEX for aptamers with B7-HTHC specificity (FIG. 6A) and aptamers with B9-THC specificity (FIG. 6B). Gel electrophoresis was used to confirm the conversion of dsDNA into desired ssDNA (data not shown).

Non-Homologous Random Recombination:

Non-homologous Random Recombination (NRR) was performed according to previous studies (Bittner J. A. et al. (2002), Nucleic acid evolution and minimization by nonhomologous random recombination, 20(10): 1024-1029). In this step, the DNA library was partially digested into smaller fragments and reassembled into a new library with aptamer sequences ranging from about 80 to 100s of bases per aptamer. The resulting aptamers vary in length and may possess multiple or shortened binding motifs, which theoretically enhances the likelihood of identifying an optimized aptamer.

Performing Non-Homologous Random Recombination (NRR) on the DNA library resulted in reduced PCR efficiency, observed by the creation of non-specific sequences (i.e., sequences that lack binding) and preference for shorter sequences in subsequent PCR steps. Thus, this step reintroduced additional variance into the library following CE SELEX aptamer selection. In the case of structure switching SELEX, elimination of fixed “structure switching regions” are not likely to be present in many of the aptamers generated from this technique, which removes them from the pool in the proceeding rounds as well. It should be noted that, in general, PCR was less efficient after the procedure, likely due to the hairpin primer design required to cap the library during the recombination, however this theory was not explored.

These issues were resolved in part by supplementing the PCR reactions with “GC enhancers” (from New England Biolabs). Primers from the original aptamer library were also included in subsequent PCR steps to amplify any sequences that were not recombined to prevent loss of any of the sequences. Gel electrophoresis was used to confirm the presence of the recombined libraries (image not shown). Gel images confirm successful recombination of many sequences greater than 200+ bases in size, albeit lack of recombination in some portions of the library were also observed (data not shown).

Structure Switching SELEX

In Stage 2, Structure Switching SELEX was used to complete aptamer selection. Structure Switching SELEX was performed by generating streptavidin modified magnetic beads with a 10-base oligomer that was complementary to a fixed “Structure Switching Region” (or “fixed sequence”) in the aptamer with a biotinylated 3′ end. Theoretically, the aptamers bound to the beads via these fixed complementary sequences.

The beads were incubated for 1 hour and washed thoroughly to remove any unbound DNA. Next, the aptamer pools were incubated with the modified beads for 1 hour at 4° C. The beads were washed 3 times with binding buffer containing 5 mM KCl, 1 mM CaCl₂), 20 mM NaCl, 100 μM MgCl₂ and 20 mM Tris (pH 7.6). Aptamers bound to beads were then incubated with the appropriate B7-HTHC or B9-THC complex for 1 hour at 4° C. and the supernatant was collected for PCR analysis.

The incubation temperature of the aptamer immuno-complexes was increased every two rounds. Round 1 and round 2 were performed at 4° C., then increased to 10° C. for round 3 and round 4, and finally 25° C. for a round 5. In round 6, negative selection was performed by incubating the aptamers with the appropriate antibody and the mismatched metabolite, i.e., B7-THC antibody was used to incubate the pool of B7-HTHC aptamers and the B9-HTHC antibody was used to incubate the pool of B9-THC aptamers.

Theoretically, when the immuno-complexes are added, aptamers demonstrating a significant structure alteration in the fixed region would release the complement modified bead. Thus, in the next step, the supernatant was discarded and the aptamers that remained on the beads were eluted by incubating the aptamers at 90° C. for 15 minutes and then collecting the supernatant for PCR.

Round 7 was performed as described for Round 6, with the exception that the aptamers were incubated with the mismatched antibody and the appropriate metabolite i.e., B9-HTHC antibody was used to incubate the pool of B7-HTHC aptamers, and the B7-THC antibody was used to incubate the pool of B9-THC aptamers. In round 8, positive aptamer selection was performed at 25° C., and the final two rounds (Rounds 9 and 10) were performed at 37° C.

Next Generation Sequencing (NGS) and Aptamer Evaluation

Following completion of aptamer selection, aptamers from the first, fifth, and tenth rounds of Structure Switching SELEX were sequenced using standard Next-Generation Sequencing (NGS) methods (AKESOgen). Of the sequences obtained by NGS, ten sequences per aptamer were tested using Capillary Electrophoresis (CE) and in a proprietary assay utilizing real-time PCR.

Results

Results from the Antibody Affinity Tests

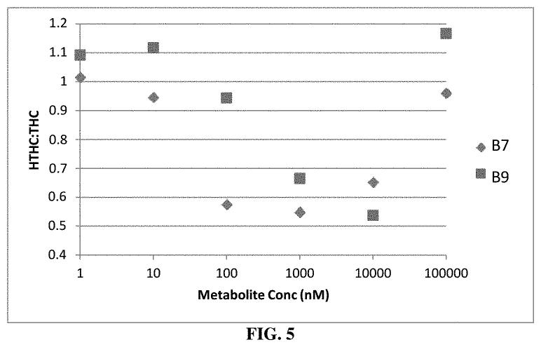

FIG. 5 is a scatter plot showing the specificity of the THC.5B7 and THC2.B9 antibodies for HTHC over THC at varying concentrations of HTHC and THC. Less reagent conversion indicates the displacement of the conjugate by the metabolite, and less overall higher affinity of the antibody for the metabolite. The HTHC and THC binding affinity was expressed as a HTHC/THC ratio. Values greater than 1 indicated that THC is preferred over HTHC, and values less than 1 indicated that HTHC is preferred. Both antibodies had binding specificity for HTHC over the THC metabolite at the 100 nM, 1000 nM, and 10,000 nM concentrations (FIG. 5). THC2.B9 antibody had stronger binding affinity for THC at the 1 nM, 10 nM and 100000 nM concentrations, while the THC.5B7 antibody had a stronger binding affinity for HTHC at these concentrations (FIG. 5). Given these results, aptamer selection was conducted for the B7-HTHC complex and the B9-THC complex.

Results from Aptamer Selection Via Structure Switching SELEX

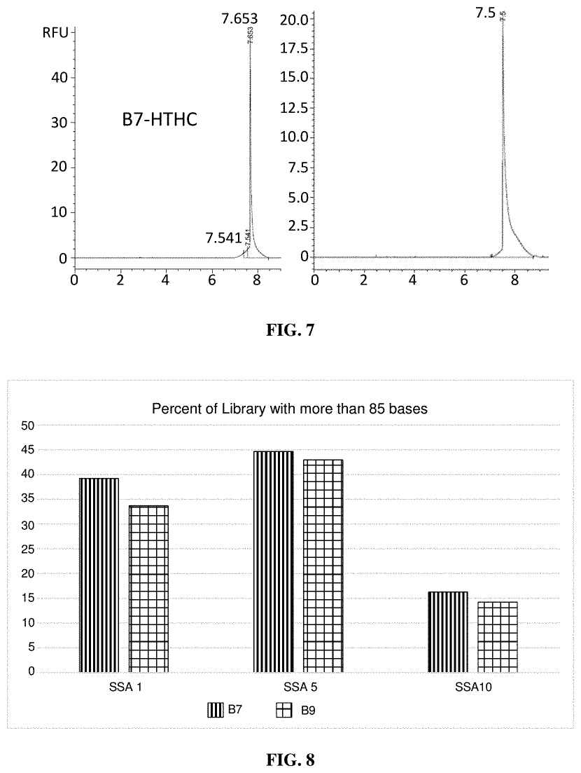

Following the completion of aptamer selection via SELEX, aptamer pools were evaluated for their binding affinities using Capillary Electrophoresis (CE). FIG. 7 is a representative electropherogram of the B7-HTHC samples. The results demonstrate a small, but prominent “ramp” prior to the bulk DNA peak, suggesting an interaction between the immune complexes and the aptamer pools (FIG. 7). However, binding of the immuno-complex was not quantifiable due to an abnormality in peak shape (FIG. 7). The abnormality in peak shape is likely due to (i) the low salt concentrations required for the CE protocol being sub-optimal for aptamer binding and (ii) the high salt concentration of the buffer used during the structure switching SELEX procedure.

Results from NGS and Evaluation of Aptamers

FIG. 8 is a bar graph showing the percent of the library containing aptamers with more than 85 bases following selection of structure switching aptamers (SSAs). Aptamers having a sequence length of 85 or more bases are significantly larger than the parent library and represent sequences that were likely generated during non-homologous random recombination (NRR). Following rounds 1 and 5 of SSA selection, 35-45% of the sequences in the aptamer pools contained 85 or more bases (FIG. 8). However, the proportion of sequences containing 85 or more bases was reduced to 15% of the aptamer pool following 10 rounds of structure switching SELEX (FIG. 8).

These results indicate that many recombined sequences lacked binding motifs, the ability to structure switch, or were non-specific for the complex compared to non-recombined sequences. The aptamers from the final round of selection were analyzed to determine candidate sequences for individual evaluation. The library was curated to include only sequences containing the fixed “structure switching” sequence. This step significantly reduced the number of candidate sequences, most of which were less than 85 bases in length. These candidate sequences were then clustered using either “FASTAptamer” or “Clustal Omega”. A total of 20 aptamers were selected, 5 aptamers from each method of clustering representing each immunocomplex. These final candidates were then processed using “mFold”, which predicts the secondary structure of single stranded DNA molecules. Candidate aptamers with a delta G>−2 were discarded and replaced with an aptamer chosen from the clustering results obtained from “Clustal Omega”. The selected candidate aptamers are listed in Table 3 and Table 4 below.

TABLE 3
Candidate aptamer sequences for antibody
B9 and THC Antibody B9 and THC

| Name | Sequence |
|---|---|
| B9TC3 | CCTGTCAGTTGCTTACCGGGCAGGCGAGTAGG |
| | ACTGCAGCGATTCTTCCTTGCGGGTGTCGGTG |
| | TAGTGTCCTTGCTCGT |
| | (SEQ ID NO: 1) |
| B9TC5 | CCTGTCAGTTGCTTACCGGGGCGAGGGACGGA |
| | GCTGCAGGATTCTTGCACTAGGTGGGGTG |
| | TGTAGTGTCCTTGCTCGT |
| | (SEQ ID NO: 2) |
| B9T191 | CCTGTCAGTTGCTTACCGGGCGGACGACATA |
| | GCCTGCAGCGATTTTCCAACACTGCGGTGGA |
| | GTAGTGTCCTTGCTCGT |
| | (SEQ ID NO: 3) |
| B9T57 | CCTGTCAGTTGCTTACCGGGGACGGCGGTGT |
| | GGCTGCAGCGATTCTTAAGAGGGCCTGGGTG |
| | TGTAGTGTCCTTGCTCGT |
| | (SEQ ID NO: 4) |
| B9TC2 | CCTGTCAGTTGCTTACCGGGGCACGGAGGAA |
| | CACTGTAGCGATTCTTGTATTCGGACCGGTGT |
| | GTAGTGTCCTTGCTCGT |
| | (SEQ ID NO: 5) |
| B9TC1 | CCTGTCAGTTGCTTACCGACACAGACGACATT |
| | ACTGCAGCCATTCTTCGCTACGTGCCCGGCTG |
| | TAGTGTCCTTGCTCGT |
| | (SEQ ID NO: 6) |
| B9T70 | CCTGTCAGTTGCTTACCGGGACGAAGGAAGA |
| | AACTGCAGCGATTCTCGTGGGGCGACGTGTA |
| | GTGTCCTTGCTCGT |
| | (SEQ ID NO: 7) |
| B9T456 | CCTGTCAGTTGCTTACCGTGTCGCCCAATGAG |
| | ACTGCAGCGATTCTTTACGGATCGGTGTCATG |
| | TAGTGTCCTTGCTCGT |
| | (SEQ ID NO: 8) |
| B9T420 | CCTGTCAGTTGCTTACCGTGCGGCGAATACG |
| | GGCTGCAGCGATTCTTTAGGGGGTCCACGGG |
| | TGTAGTGTCCTTGCTCGT |
| | (SEQ ID NO: 9) |
| B9T46 | CCTGTCAGTTGCTTACCGGCACGGTGATCGTA |
| | CTGCAGGATTCTTCATTTCGCCGCGTCGTGTA |
| | GTGTCCTTGCTCGT |
| | (SEQ ID NO: 10) |

TABLE 4
Candidate sequences for antibody B7 and
HTHC metabolite Antibody B7 and HTHC

| Name | Sequence |
|---|---|
| B7H357 | CCTGTCAGTTGCTTACCGGGCCGAGCGTAC |
| | GGACTGCAGCGATTCTTGGTGACCAGCCGG |
| | GGAGTAGTGTCCTTGCTCGT |
| | (SEQ ID NO: 11) |
| B7HC3 | CCTGTCAGTTGCTTACCGGGCGGAGACGGA |
| | AGACTGCAGCAATTCTTGGTGCTGTGTGTG |
| | GCTGTAGTGTCCTTGCTCGT |
| | (SEQ ID NO: 12) |
| B7HC4 | CCTGTCAGTTGCTTACCGGGCACGAGTACG |
| | AGACTGCAGGATTCTTCTACACTGCCCCCCA |
| | TGTAGTGTCCTTGCTCGT |
| | (SEQ ID NO: 13) |
| B7HC5 | CCTGTCAGTTGCTTACCGGGCAGCGGTGCA |
| | AGACTGCAGGATTCTTCAGAGGCGGGCCGC |
| | GTGTAGTGTCCTTGCTCGT |
| | (SEQ ID NO: 14) |
| B7H341 | CCTGTCAGTTGCTTACCGGGCACGATGCAA |
| | ATACTGCAGCGATTCTTGATACCGGTCGCGT |
| | GTGTAGTGTCCTTGCTCGT |
| | (SEQ ID NO: 15) |
| B7H102 | CCTGTCAGTTGCTTACCGACGAAGGCGCTG |
| | AAACTGCAGCGATTCTTGCGGTGTACTGAG |
| | TGTGTAGTGTCCTTGCTCGT |
| | (SEQ ID NO: 16) |
| B7HC1 | CCTGTCAGTTGCTTACCGGCCCAGGTGTAGT |
| | AACTGCAGGGATTCTTAACGTAGCTACCGG |
| | GTGTAGTGTCCTTGCTCGT |
| | (SEQ ID NO: 17) |
| B7HC2 | CCTGTCAGTTGCTTACCGGGCGGCACATGC |
| | TAGCTGCAGCGATTCTTGTATTAGGTGCGG |
| | CGTGTAGTGTCCTTGCTCGT |
| | (SEQ ID NO: 18) |
| B7H422 | CCTGTCAGTTGCTTACCGCAGGGCGTCAGG |
| | GACCTGCAGCGATTCTTGGTGTAGGGGGCC |
| | GCTGTAGTGTCCTTGCTCGT |
| | (SEQ ID NO: 19) |
| B7H338 | CCTGTCAGTTGCTTACCGGCACGGGAAGTT |
| | GGACTGCAGCGATTCTTTCGACGCCCGGCC |
| | CCGTGTAGTGTCCTTGCTCGT |
| | (SEQ ID NO: 20) |

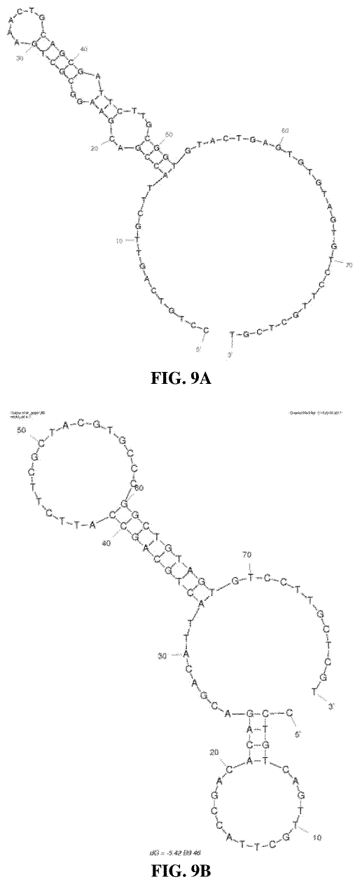

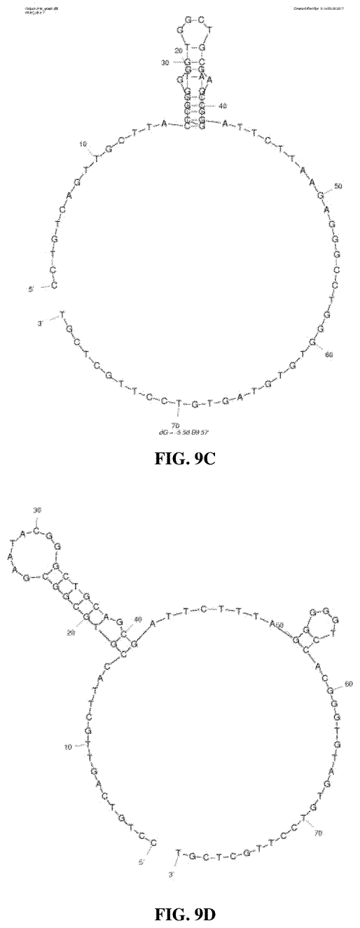

Examination of the secondary structures of the candidate aptamers revealed alterations in the fixed structure switching area for each candidate aptamer. These results indicate that the aptamers were in an unfolded state and bound to the complementary sequence. The results also suggest that the candidate aptamers were released when the immuno-complexes were added, thereby inducing a structural change in the aptamer within the structure switching region. FIGS. 9A-9D are graphical representations of the secondary structures of several of the candidate aptamers. The structure switching region extend from base 34 to base 45. FIG. 9A contains SEQ. ID NO: 6. FIG. 9B contains the sequence 5′ CCTGTCAGTTGCTTACCCGGGGTGGTGGCT GCGAAGCCGGGATTCTTAAGAGGGCCTGGGTGTGTAGTGTCCTTGCT CGT 3′ (SEQ ID NO:31). FIG. 9C contains the sequence 5′ CCTGTCAGTT GCTTACCGTGCGGCGAATACGGGCTGCAGCGATTCTTTAGGGGGTCC ACGGGTGTAGTGTCCTTGCTCGT 3′ (SEQ ID NO:32). FIG. 9D contains the sequence 5′ CCTGTCAGTTGCTTACCGTGTAGTGTCCTTGCTCGTCC TGTCAGTTGCTTACCGTGTAGTGTCCTGCTCGT 3′ (SEQ ID NO: 33).

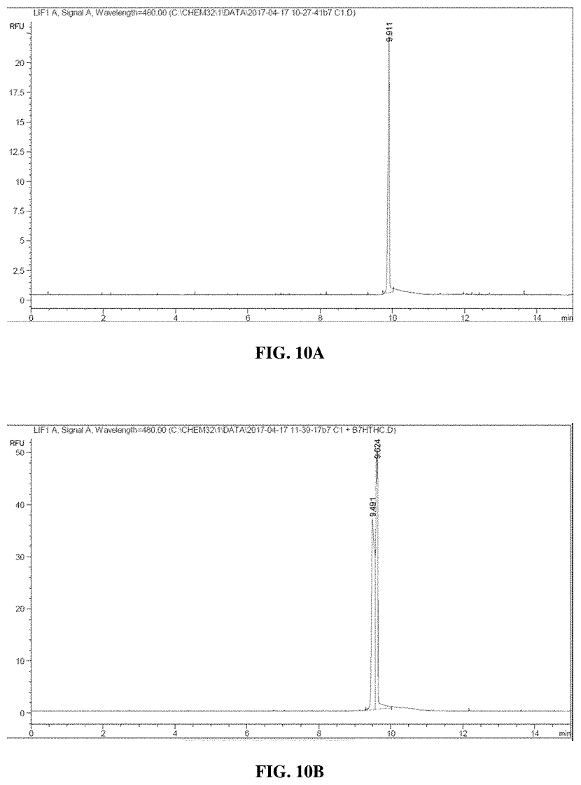

The candidate aptamers were initially tested using capillary electrophoresis. Of the 10 B7-HTHC aptamers, 3 were identified as candidate binders. However, the peaks for the 3 candidate binders were not well separated from the bulk DNA peak in a similar manner to what was observed with the bulk libraries prior to sequencing. This is likely due to the difference between the composition of the buffer required for capillary electrophoresis and the composition of the buffer used during the SELEX selection process. FIGS. 10A and 10B are electropherograms showing the peak shifts associated with the introduction of the immune complex in aptamer B7C1.

Binding selectivity was tested in varying salt conditions to determine optimal conditions for selection. Using a proprietary binding assay, the aptamers were allowed to incubate with immune complexes that were anchored to a solid support. After the incubation period, the immune complexes were washed, and the amount of aptamer binding was assayed via qPCR. Increased aptamer binding to the immune complex allowed faster detection of DNA amplification.

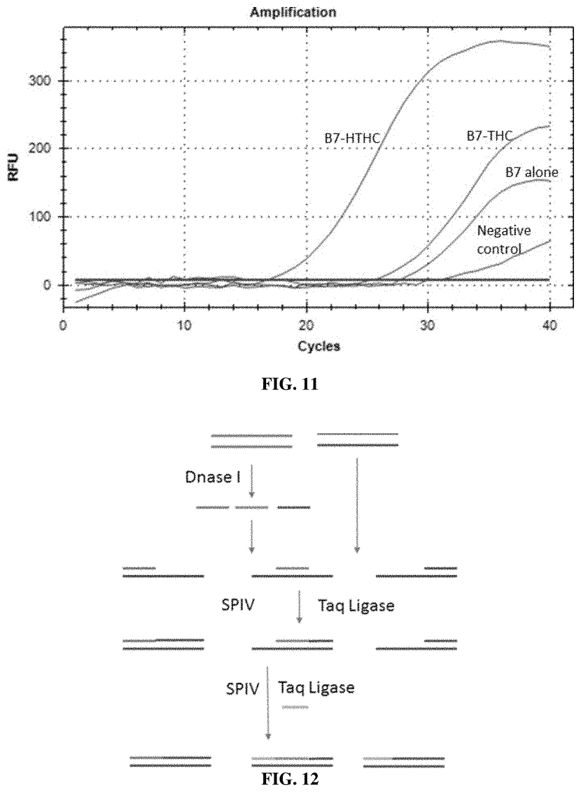

Of the aptamers tested, only one aptamer satisfied the following requirements: (1) binding to the correct immune complex, (2) no binding to the antibody alone, and (3) no binding to the antibody and the incorrect metabolite. FIG. 11 is a qPCR amplification curve showing the binding of Aptamer B7C4 with the immune complex B7-HTHC, the immune complex B7-THC, the B7 antibody alone, and a negative control. Theoretically, every additional cycle indicates 2 times fewer bound aptamer molecules. Aptamer B7C4 demonstrated significant binding to the B7-HTHC immune complex and minimal binding to either the B7 antibody alone or B7-THC immune complex (FIG. 11). While the absolute binding constant of aptamer B7C4 cannot be determined using this assay, the results from this assay demonstrate the specificity of the B7C4 aptamer for the B7-HTHC immune complex (FIG. 11).

Discussion

An aptamer that selectively binds to the B7-HTHC immune complex was identified using a combination of capillary electrophoresis and bead-based Structure Switching SELEX. The inclusion of non-homologous random recombination (NRR) did not appear to have played a significant role in improving the aptamer pool and most sequences that underwent NRR were removed during the SELEX process. Further, following sequencing of the libraries, most of the sequences containing the critical structure switching sequence were of length 85 bases or less. This indicated that the sequences containing the critical structure switching region did not undergo NRR. Thus, in these experiments, NRR did not provide an added benefit to the SELEX process.

Next Generation Sequencing (NGS) also demonstrated more heterogeneity in the library than was expected; likely resulting from using multiple SELEX methods for aptamer selection. Target complexity may have also contributed to the heterogeneity of the candidate pools. Analysis of the libraries yielded 10 candidate aptamers which were assessed for binding to each immuno-complex. These candidate aptamers demonstrated inferior separation between complex and bulk DNA following capillary electrophoresis, with only B7-HTHC aptamers having acceptable binding efficiency. Further testing using a qPCR-based method suggested that only one aptamer, notably aptamer B7C4 had acceptable binding and specificity for its target.

### Example 3: Optimization of the B7C4-HTHC Aptamer

Aptamer B7C4-HTHC (SEQ ID NO:13) was successfully created and evaluated in Example 2. Aptamer B7C4-HTHC (SEQ ID NO:13) can differentiate between two similar antibodies bound to HTHC, and the B7 antibody bound to THC (as opposed to the metabolite, HTHC). To differentiate between binding of the B7C4-HTHC aptamer (SEQ ID NO:13) to the B7 antibody and the B7-HTHC immunocomplex, ultra-sensitive methods were required. This is likely due to significant background binding of the B7C4-HTHC aptamer (SEQ ID NO:13) to the antibody alone, thereby limiting its sensitivity to increased levels of the HTHC metabolite. This suggests that the B7C4-HTHC aptamer (SEQ ID NO:13) recognizes portions of both the B7 antibody and the HTHC metabolite. Thus, while the B7C4-HTHC aptamer (SEQ ID NO:13) demonstrates high affinity for the combined B7-HTHC immunocomplex, it still has residual binding affinity for the unbound B7 antibody.

In this non-limiting example, experiments were conducted to refine the binding specificity of the B7C4-HTHC aptamer (SEQ ID NO:13) to reduce non-specific binding to the B7 antibody.

Generation and Evaluation of an Aptamer Library Containing Derivatives of the B7-HTHC Aptamer (SEQ ID NO:13)

In order to differentiate between the B7 antibody and the B7-HTHC immunocomplex, ultra-sensitive probes are required. To reduce non-specific binding to the B7 antibody alone, the sequence of the B7C4-HTHC aptamer (SEQ ID NO:13) was refined. This process was accomplished by mutating the known sequence of the B7C4-HTHC aptamer (SEQ ID NO:13) such that each base had either 75% or 82% chance to be synthesized as the original base. This randomization procedure generated new aptamers that were derivatives of the B7-HTHC aptamer (SEQ ID NO:13). Theoretically, while some derivatives of the mutated B7-HTHC aptamer (SEQ ID NO:13) would lose the ability to bind to the target immunocomplex, other derivatives would increase the specificity for the target immunocomplex. The aptamer libraries were subjected multiple rounds of selection (as described in Example 2) to identify advantageous derivatives of the B7-HTHC aptamer (SEQ ID NO:13). Also, midway through the selection steps, the aptamer library was shuffled to further increase the variance of the library and permit the recombination of advantageous mutations present on separate aptamers into a single sequence.

Two libraries were synthesized based on the sequence of the B7C4-HTHC aptamer (SEQ ID NO:13) such that each base in the variable sequence region of the aptamer had either a 75% or 82% possibility of conforming to the parent sequence B7C4-HTHC aptamer (SEQ ID NO:13). These libraries were then mixed for selection purposes.

Selection of B7-HTHC Aptamer Derivatives Containing Advantageous Mutations

In round 1 of the SELEX process, a positive selection was performed to remove mutated aptamers that lost the ability to bind to the B7-HTHC immunocomplex. The mutated aptamers were attached to magnetic beads via a fixed “structure-switching region” construct incorporated into the original sequence of structure-switching region of the parent B7C4-HTHC aptamer (SEO ID NO:13). To accomplish this, streptavidin-coated magnetic beads and a compliment DNA sequence labeled with biotin were used. The mutated aptamers base-paired with the bead-bound compliment sequence and were released upon binding the immunocomplex. A premixed solution of the B7 antibody and HTHC metabolite (1:100) was added to the bead-bound aptamers and the mixture was incubated for 1 hour. Next, the mutated aptamers that were bound to the immunocomplex were collected and aptamers that remained bound to the beads were also separately collected for analysis via diagnostic PCR.

Following positive selection, most of the mutated aptamers in the aptamer library separated from the beads (data not shown). A small number of the mutated aptamers remained bound to the beads (data not shown), indicating their inability to make a structure switch or loss of their ability to recognize the target immunocomplex. This result indicated that even with incorporated mutations, most of the B7C4-HTHC-derived aptamers recognized and bound to the B7-HTHC immunocomplex.

For subsequent rounds of selection, the mutated aptamer sequences collected in the positive selection step were degraded into ssDNA. The following 5 rounds (rounds 2-6) of selection were carried out in a similar manner as described for positive selection, but negative selection was performed instead of positive selection. For these rounds of selection, the aptamers were bound to the beads as described above for positive selection, but only the B7 antibody (without the HTHC metabolite) was added to the bead-bound aptamers. Aptamers that bound to the B7 antibody alone were released from the beads and were discarded. Aptamers that were not bound to the B7 antibody and that remained bound to the beads were collected and subjected to further rounds of selection.

Following completion of the first set of aptamer selections, the aptamer pool was clipped and reshuffled by dividing the pool of DNA sequences into two samples. The first sample contained 90% of the DNA sequences (Sample A) and the second sample contained 10% of the DNA sequences (Sample B). Sample A containing 90% of the DNA sequences was digested using varying concentrations of DNAse I. DNAse I degraded the full-length sequences of DNA into shorter fragments, the size of which was dependent on the amount of DNAse I used and the incubation time.

Increasing the volume of DNAse I used for digestion of the Sample A fragments from 0.00125 μL to 0.125 μL (in 100 μL of dsDNA) resulted in a concentration-dependent decrease in fragment size as well as a reduction in the DNA signal obtained from gel-electrophoresis analysis (data not shown). This reduction in overall signal was likely due to the digestion of the DNA sequences into shorter single nucleotide fragments.

The fragmented DNA sample was mixed with the second sample of undigested DNA, along with Sulfolobus DNA polymerase IV and Taq DNA Ligase. In the mixture, undigested DNA hybridized with small fragments of DNA and served as a template to extend the fragments, thereby resulting in recombination of the B7-HTHC aptamers.

After 5 cycles of recombination, primers were added to the mixture to allow conversion of the shorter fragments to full-length sequences and normal PCR amplification was performed to increase the amount of DNA for further rounds of aptamer selection. Compared to the previously used recombination design for B7C5-HTHC aptamers, this change in the recombination approach was an improvement in the experimental design because it generated DNA that was of similar size and structure as the parent DNA sequence. The previous method of DNA recombination produced sequences that were significantly larger than the parent DNA (85 or more bases) with significantly lower binding affinity for the immunocomplexes.

FIG. 12 is a graphical representation of the recombination steps. Analysis of the DNA fragments using gel electrophoresis revealed that the most of the recombinant DNA fragments were similar size as the parent library, although some larger sequences were observed as smears on the gel (data not shown).

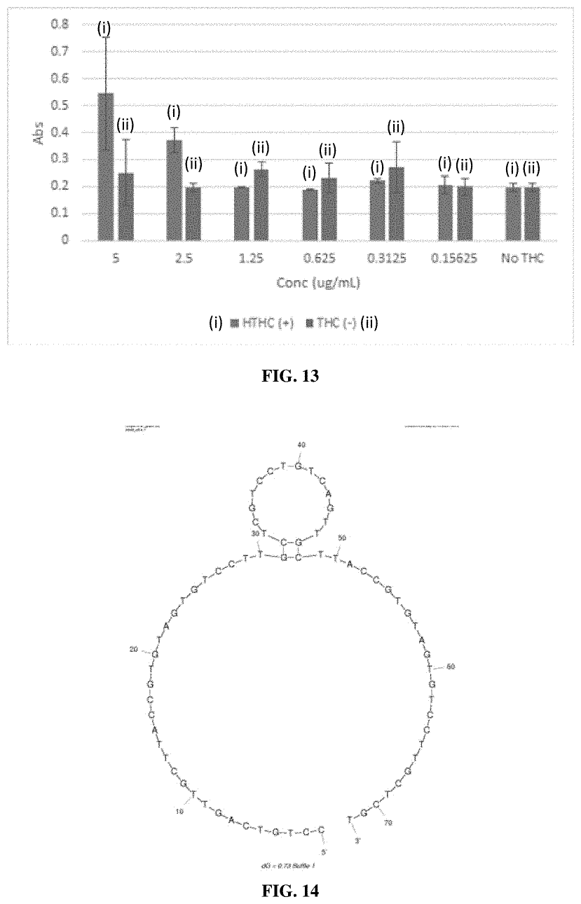

Next, the aptamer pool was subjected to two rounds of positive selection as described above to remove any sequences that lost their ability to make a structural switch and/or bind the immunocomplex or were otherwise compromised. These positively selected aptamers were then subjected to five rounds of negative selection to identify aptamers with reduced binding to the B7 antibody, followed by two final rounds of positive selection. The final aptamer pool was assayed for its ability to bind the B7-HTHC immunocomplex using an ELISA-like assay prior to next generation sequencing (NGS) analysis. FIG. 13 is a bar graph showing the absorption signal from the sandwich type ELISA assay at different concentrations of HTHC or THC. FIG. 13 consolidates the results from two experiments, one in which HTHC is present (HTHC+) and a second experiment in which THC is absent (THC−). As shown in FIG. 13, there is a dose dependent response in the binding of the selected derived-aptamers to the B7-HTHC immunocomplex, but not the B7-THC immunocomplex. The derived-aptamer pool did not show any response to the presence of the B9 antibody regardless of the presence of HTHC.

Analysis of Data Obtained from Next Generation Sequencing (NGS)

Bioinformatics analyses was conducted on the aptamer sequences to remove non-aptamer sequences using the 5′ primer sequence and Illumina adapter sequences as a guide. Initial analysis of the data revealed that a large proportion of the identified sequences were highly disordered and lacked a definitive structure, often with a positive ΔG value.

Analysis of the most populous NGS sequence (FIG. 14) revealed that it did not possess the constant “structure-switching sequence” that enables aptamer binding and signal release. Further analysis revealed that none of the sequences lacking structure contained this critical feature, despite being fully conserved (100%) with the doped (partially randomized) libraries. These unstructured sequences were determined to be multiple primer regions annealed together, likely during the shuffling phase of the selection. Despite their lack of affinity to the immunocomplex, their ability to self-prime and to bind multiple primers during a single round of PCR resulted in their favored amplification. To eliminate these sequences from the data set, the data was filtered to eliminate sequences which did not contain the conserved structure switching region with at least 70% fidelity.

An identical copy of the B7C4-HTHC aptamer was the most populous aptamer observed in the NGS dataset, indicating that the B7C4-HTHC aptamer is a relatively selective and sensitive aptamer (FIG. 15A, SEQ ID NO:13). Further analysis of the data using phylogenetic methods revealed three major families of sequences (FIGS. 15B-15D). Family 1 contained a mutation in the second random region 3′ to the structure-switching sequence (FIG. 15B, SEQ ID NO:34). These mutations resulted in an alteration to the stem loop of the aptamers and represent approximately 85% of the sequences in the data set (FIG. 15B, SEQ ID NO:34). Family 2 accounted for approximately 10% of the sequences in the data set and contained mutations in the random region which formed base pairs with the structure-switching region (FIG. 15C, SEQ ID NO:26). Mutations here altered the shape and strength of the structure-switching base pairs (FIG. 15C, SEQ ID NO:26). Similar to family 1, family 3 contained mutations in the second random region, but the mutations occurred in areas that do not appear to have any secondary structure (FIG. 15D, SEQ ID NO:28).

Of note is that most aptamers only possessed a single mutation; some had two mutations, but only a single aptamer was identified with more than two mutations. This result suggested that only minor alterations to the parent aptamer were necessary and additional mutations likely resulted in a loss of function. Of particular note is the mutated aptamer in FIG. 15E (SEQ ID NO:17). This aptamer was highly mutated, with ⅔ of the bases in the 3′ random region (associated with structure switching) different from the parent B7C4-HTHC aptamer (FIG. 15E, SEQ ID NO:17). The second random region was also highly mutated. This sequence was the second most populous sequence in the filtered NGS dataset.

Sandwich Type Assay with the Selected Derived B7-HTHC Aptamers

Based on the NGS results (the prevalence of each sequence, the structure, and the phylogenetic clustering of the sequences), 11 sequences were chosen for subsequent testing using sandwich type assays. Table 5 summarizes these sequences.

The 11 aptamers were synthesized with a 5′ biotin such that they could be detected in ELISA-type assays. The aptamers were tested for their ability to bind to the target in two ways. In both assays, the antibody was coated onto the walls of a microtiter plate. In the first assay, HTHC was added to the antibody. After washing away unbound HTHC, the aptamers were added. In the second assay, the aptamers were added to the wells with HTHC simultaneously. In both assays, after washing away unbound aptamer, the plates were incubated with HRP-streptavidin, and then assayed for the amount of aptamer that remained bound to the immunocomplex using a colorimetric TMB assay.

TABLE 5
Sequences of Selected Derived B7-HTHC Aptamers

| SEQ | | |
|---|---|---|
| ID | | |
| NO. | Name | Sequence (5′ to 3′) |
| 13 | 368 | CCTGTCAGTTGCTTACCGGGCACGAGTACGAGACTGCAGGA |
| | | TTCTTCTACACTGCCCCCCATGTAGTGTCCTTGCTCGT |
| 21 | 368.2 | CCTGTCAGTTGCTTACCGGGCACGAGTACGAGACTGCAGGA |
| | | TTCTTCTACACTGCTCCCCATGTAGTGTCCTTGCTCGT |
| 22 | 154 | CCTGTCAGTTGCTTACCGGCCCAGGTGTAGTAACTGCAGGG |
| | | ATTCTTAACGTAGCTACCGGGTGTAGTGTCCTTGCTCGT |
| 23 | 444 | CCTGTCAGTTGCTTACCGGGCACGAGTACGAGACTGCAGGA |
| | | TTCTTCTACACTGCGCCCCATGTAGTGTCCTTGCTCGT |
| 24 | 489 | CCTGTCAGTTGCTTACCGGGCACGAGTACGAGACTGCAGGA |
| | | TTCTTCTACACTGGCCCCCATGTAGTGTCCTTGCTCGT |
| 25 | 493 | CCTGTCAGTTGCTTACCGGGCACGAGTACGAGACTGCAGGA |
| | | TTCTTCTACACTGCACCCCATGTAGTGTCCTTGCTC |
| 26 | 815 | CCTGTCAGTTGCTTACCGGGCACGAGTACTAGACTGCAGGA |
| | | TTCTTCTACACTGCCCCCCATGTAGTGTCCTTGCTCGT |
| 27 | 732 | CCTGTCAGTTGCTTACCGGCCACGAGTACGAGACTGCAGGA |
| | | TTCTTCTACACTGCCCCCCATGTAGTGTCCTTGCTCGT |
| 28 | 568 | CCTGTCAGTTGCTTACCGGGCACGAGTACGAGACTGCAGGA |
| | | TTCTTTTACACTGCCCCCCATGTAGTGTCCTTGCTCGT |
| 29 | 550 | CCTGTCAGTTGCTTACCGGGCACGAGTACGAGACTGCAGGA |
| | | TTCTTCTACACTGCCCCACATGTAGTGTCCTTGCTCGT |
| 30 | 333 | CCTGTCAGTTGCTTACCGGGCACGAGTACGAGACTGCAGGA |
| | | TTCTTCTACACTGCCCCCCATGTAGTGTCCTTGCTCGTGTA |
| | | GTGTCCTTGCTCGT |

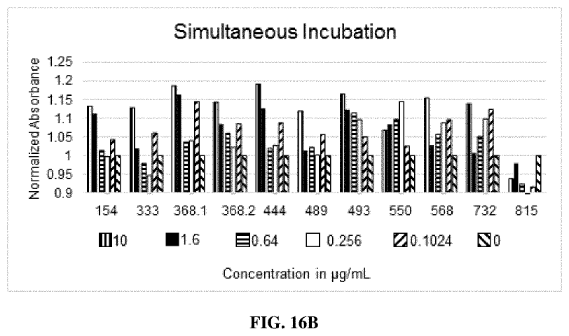

The results of the sandwich type assays are shown in FIGS. 16A and 16B. It is evident that some of DNA aptamers responded better under simultaneous incubation (FIG. 16B) whereas others responded better under separate incubation (FIG. 16A). Separate incubation also generally resulted in higher signal intensities, perhaps due to reduced displacement from free HTHC in solution (FIG. 16A). Many of the aptamers displayed an increase in binding with low HTHC content in the simultaneous incubation assay, further suggesting some kinetic effects were involved (FIG. 16B). Nearly all aptamers displayed the ability to detect the immunocomplex at the microgram per milliliter level, while some were able to detect down to the high nanogram level, depending on the assay design. For example, aptamer 815 (SEQ ID NO: 26) displayed high binding when incubated separately (FIG. 16A), but lost this ability during simultaneous incubation (FIG. 16B). Aptamer 444 (SEQ ID NO: 23) showed high sensitivity in separate incubation (FIG. 16A), but exhibited reduced sensitivity in simultaneous incubation (FIG. 16B). In contrast, aptamer 493 (SEQ ID NO: 25) demonstrated the opposite behavior compared to that of aptamer 444 (SEQ ID NO: 23). Aptamer 368.2 (SEQ ID NO: 21) displayed significant binding in both assay designs.

Taken together, the results showed that the aptamers derived from B7C4-HTHC could detect the difference between unbound antibody and the immunocomplex with varying levels of HTHC. These aptamers can be utilized in a variety of sandwich-type assays for HTHC, which are not possible using traditional antibody-based assays.

Modifications and variations of the methods and reagents described herein will be obvious to those skilled in the art from the foregoing detailed description. Such modifications and variations are intended to come within the scope of the appended claims. All references cited herein are specifically incorporated by reference.

I claim:

1. A non-competitive assay method for quantitatively measuring the amount of an analyte in a biological sample from a subject, comprising:
a) reacting the biological sample with
i) a binding agent that selectively binds the analyte to form a capture complex of the binding agent and analyte, and
(ii) a capture agent that selectively binds to the capture complex but not the free analyte to form a sandwich complex of the binding agent, capture agent, and analyte, and
b) measuring sandwich complex formation;
wherein the analyte has a molecular weight of less than 2,000 Daltons,
wherein the binding agent is an antibody of the analyte,
wherein the amount of sandwich complex formation is directly related to the amount of the analyte in the sample.

2. The non-competitive assay method of claim 1, wherein the capture agent comprises any one of SEQ ID Nos 13, or 21-35 or a derivative or mutant thereof comprising a sequence that has between about 70% and 100% sequence identity to any one of SEQ ID NOs 13, or 21-35, to the variable sequence region of any one of SEQ ID NOs 13, or 21-35, or to the structure-switching region of any one of SEQ ID NOs 13, or 21-35, optionally, wherein the binding agent is linked to a first detectable label, and the capture agent is linked to a second detectable label, wherein the first and second detectable labels are fluorescent molecules with distinct excitation and emission wavelength combinations or form a fluorescence resonance energy transfer (FRET) donor-acceptor pair.

3. The non-competitive assay method of claim 1, wherein the binding agent or capture agent comprises a fluorophore and quencher pair, wherein formation of the sandwich complex results in detectable quenching or unquenching of the fluorophore.

4. The non-competitive assay method of claim 1, wherein the analyte is a hormone, drug, or drug metabolite and optionally, wherein the analyte is selected from the group consisting of morphine, codeine, thebaine, heroin, hydromorphone, hydrocodone, oxycodone, oxymorphone, desomorphine, nicomorphine, propoxyphene, dipropanoylmorphine, benzylmorphine, ethylmorphine, buprenorphine, fentanyl, pethidine, meperidine, methadone, tramadol, dextropropoxyphene, noroxycodone, norhydrocodone, THC, THC-COOH, 11-OH-THC, cannabidiol, amphetamines, cocaine and analogs thereof, benzodiazepines, hallucinogens, nicotine, metabolites thereof, and combinations thereof.

5. The non-competitive assay method of claim 1, wherein the assay is a lateral flow immunoassay comprising:
a) optionally adding the binding agent to the biological sample;
b) applying the biological sample to a membrane strip comprising an application point, an optional conjugation zone, a capture zone, and an absorbent zone, wherein the conjugation zone comprises the binding agent, wherein the capture zone comprises the capture agent immobilized in or on the membrane strip, wherein the biological sample is applied to the application point;
c) optionally maintaining the membrane strip under conditions that allow the analyte present in the biological sample to move by capillary action through the membrane strip to the conjugation zone and to allow binding of the binding agent to the analyte to form the capture complex;
d) maintaining the membrane strip under conditions that allow the capture complex to move by capillary action through the membrane strip to the capture zone and to allow binding of the capture agent to the capture complex to form the sandwich complex;
e) further maintaining the membrane strip under conditions which allow movement of the binding agent not immobilized in the capture zone into the absorbent zone; and
f) determining the amount of the sandwich complex in the capture zone, wherein the amount of the analyte in the sample is directly correlated to the amount of the sandwich complex present in the capture zone.

6. The non-competitive assay method of claim 5, wherein: (a) the membrane strip comprises a material selected from the group consisting of cellulose, cellulose nitrate, cellulose acetate, glass fiber, nylon, polyelectrolyte, acrylic copolymer, and polyethersulfone; (b) the binding agent is linked to a first detectable label, and the capture agent is linked to a second detectable label, and wherein the amount of the sandwich complex is determined as a ratio of the amount of first detectable label detected in the capture zone to the amount of a control detectable label detected in the capture zone, optionally wherein the control detectable label is the second detectable label; and/or (c) the capture agent is conjugated to particles trapped within the membrane strip and localized to a capture line within the capture zone.

7. The non-competitive assay method of claim 1, wherein the membrane strip comprises a single layer Fusion 5 material.

8. The non-competitive assay method of claim 6, wherein: (a) a control detectable label is in or on the particles; and/or (b) the amount of the binding agent detected in the capture line is normalized to the amount of a control binding agent that specifically binds a control analyte detected in a control capture line.

9. The method of claim 8, wherein the control analyte is added to the biological sample before the sample is administered to the application point of the membrane strip.

10. The method of claim 7, wherein the amount of the analyte in the biological sample is determined by plotting the amount of the binding agent detected against a response surface calculated from a plurality of analyte standards.

11. The method of claim 10, wherein the response surface is adjusted using internal controls.

12. The method of claim 1, wherein the capture agent is conjugated on magnetic beads.

13. A kit for performing a non-competitive assay for an analyte selected from the group consisting of a hormone, drug, and drug metabolite having a molecular weight of less than 2,000 Daltons,
wherein the kit comprises a membrane strip comprising an application point, a capture zone, and an absorbent zone,
wherein the capture zone comprises a capture agent that selectively binds to a binding agent-analyte complex but not the free analyte, immobilized in the capture zone of the membrane strip, and
wherein the binding agent is an antibody of the analyte,
wherein the capture agent is a nucleic acid aptamer selected from a synthetic oligonucleotide library using the capture complex as a target ligand.

14. The kit of claim 13, wherein: (a) the membrane strip further comprises a conjugation zone, wherein the conjugation zone comprises the binding agent; and/or (b) the kit further comprising a sample collection apparatus, wherein the sample collection apparatus contains the binding agent the capture zone comprises an immobilized control analyte.

15. The kit of claim 13, wherein the nucleic acid aptamer is selected from the synthetic oligonucleotide library via one or more positive selection cycles comprising:
(a) mixing the synthetic oligonucleotide library with the capture complex under conditions that allow for binding of oligonucleotides in the synthetic oligonucleotide library to the capture complex;
(b) isolating oligonucleotides that bind to the capture complex;
(c) amplifying the oligonucleotides from step (b) to generate a library of amplified oligonucleotides; and
(d) repeating steps (a)-(c) for a plurality of rounds to obtain nucleic acid aptamers capable of binding to the capture complex, wherein the synthetic oligonucleotide library in step (a) is substituted by the library of amplified oligonucleotides from the previous round.

16. The kit of claim 15, further comprising one or more negative selection cycles comprising:
(a) mixing the library of amplified oligonucleotides from the previous round with the binding agent or analyte;
(b) isolating oligonucleotides that do not bind to the binding agent or analyte;
(c) amplifying the oligonucleotides from step (b) to generate a library of amplified oligonucleotides; and
(d) repeating steps (a)-(c) for a plurality of rounds to obtain nucleic acid

17. The kit of claim 13, wherein the capture agent comprises any one of SEQ ID NOs 1-35 or a derivative or mutant thereof comprising a sequence that has between about 70% and 100% sequence identity to any one of SEQ ID NOs 1-30, to the variable sequence region of any one of SEQ ID NOs 1-35, or to the structure-switching region of any one of SEQ ID NOs 1-35.

18. A single-stranded oligonucleotide, having a sequence comprising any one of SEQ ID NOs 1-35 or a derivative or mutant thereof comprising a sequence that has between about 70% and 100% sequence identity to any one of SEQ ID NOs 1-35, to the variable sequence region of any one of SEQ ID NOs 1-35, or to the structure-switching region of any one of SEQ ID NOs 1-35.

19. The oligonucleotide of claim 18, wherein the oligonucleotide is a DNA oligonucleotide.


SEQUENCE LISTING (35 sequences)

SEQ ID NO: 1 - DNA, 80 bp, Artificial Sequence
  Synthetic nucleic acid aptamer sequence for the B9-THC immunocomplex (B9TC3)
cctgtcagttgcttaccgggcaggcgagtaggactgcagcgattcttccttgcgggtgtcggtgtagtgtccttgctcgt

SEQ ID NO: 2 - DNA, 79 bp, Artificial Sequence
  Synthetic nucleic acid aptamer sequence for the B9-THC immunocomplex (B9TC5)
cctgtcagttgcttaccggggcgagggacggagctgcaggattcttgcactaggtggggtgtgtagtgtccttgctcgt

SEQ ID NO: 3 - DNA, 79 bp, Artificial Sequence
  Synthetic nucleic acid aptamer sequence for the B9-THC immunocomplex (B9T191)
cctgtcagttgcttaccgggcggacgacatagcctgcagcgattttccaacactgcggtggagtagtgtccttgctcgt

SEQ ID NO: 4 - DNA, 80 bp, Artificial Sequence
  Synthetic nucleic acid aptamer sequence for the B9-THC immunocomplex (B9T57)
cctgtcagttgcttaccggggacggcggtgtggctgcagcgattcttaagagggcctgggtgtgtagtgtccttgctcgt

SEQ ID NO: 5 - DNA, 80 bp, Artificial Sequence
  Synthetic nucleic acid aptamer sequence for the B9-THC immunocomplex (B9TC2)
cctgtcagttgcttaccggggcacggaggaacactgtagcgattcttgtattcggaccggtgtgtagtgtccttgctcgt

SEQ ID NO: 6 - DNA, 80 bp, Artificial Sequence
  Synthetic nucleic acid aptamer sequence for the B9-THC immunocomplex (B9TC1)
cctgtcagttgcttaccgacacagacgacattactgcagccattcttcgctacgtgcccggctgtagtgtccttgctcgt

SEQ ID NO: 7 - DNA, 76 bp, Artificial Sequence
  Synthetic nucleic acid aptamer sequence for the B9-THC immunocomplex (B9T70)
cctgtcagttgcttaccgggacgaaggaagaaactgcagcgattctcgtggggcgacgtgtagtgtccttgctcgt

SEQ ID NO: 8 - DNA, 80 bp, Artificial Sequence
  Synthetic nucleic acid aptamer sequence for the B9-THC immunocomplex (B9T456)
cctgtcagttgcttaccgtgtcgcccaatgagactgcagcgattctttacggatcggtgtcatgtagtgtccttgctcgt

SEQ ID NO: 9 - DNA, 80 bp, Artificial Sequence
  Synthetic nucleic acid aptamer sequence for the B9-THC immunocomplex (B9T420)
cctgtcagttgcttaccgtgcggcgaatacgggctgcagcgattctttagggggtccacgggtgtagtgtccttgctcgt

SEQ ID NO: 10 - DNA, 78 bp, Artificial Sequence
  Synthetic nucleic acid aptamer sequence for the B9-THC immunocomplex (B9T46)
cctgtcagttgcttaccggcacggtgatcgtactgcaggattcttcatttcgccgcgtcgtgtagtgtccttgctcgt

SEQ ID NO: 11 - DNA, 80 bp, Artificial Sequence
  Synthetic nucleic acid aptamer sequence for the B7-HTHC immunocomplex (B7H357)
cctgtcagttgcttaccgggccgagcgtacggactgcagcgattcttggtgaccagccggggagtagtgtccttgctcgt

SEQ ID NO: 12 - DNA, 80 bp, Artificial Sequence
  Synthetic nucleic acid aptamer sequence for the B7-HTHC immunocomplex (B7HC3)
cctgtcagttgcttaccgggcggagacggaagactgcagcaattcttggtgctgtgtgtggctgtagtgtccttgctcgt

SEQ ID NO: 13 - DNA, 79 bp, Artificial Sequence
  Synthetic nucleic acid aptamer sequence for the B7-HTHC immunocomplex (B7HC4)
cctgtcagttgcttaccgggcacgagtacgagactgcaggattcttctacactgccccccatgtagtgtccttgctcgt

SEQ ID NO: 14 - DNA, 79 bp, Artificial Sequence
  Synthetic nucleic acid aptamer sequence for the B7-HTHC immunocomplex (B7HC5)
cctgtcagttgcttaccgggcagcggtgcaagactgcaggattcttcagaggcgggccgcgtgtagtgtccttgctcgt

SEQ ID NO: 15 - DNA, 80 bp, Artificial Sequence
  Synthetic nucleic acid aptamer sequence for the B7-HTHC immunocomplex (B7H341)
cctgtcagttgcttaccgggcacgatgcaaatactgcagcgattcttgataccggtcgcgtgtgtagtgtccttgctcgt

SEQ ID NO: 16 - DNA, 80 bp, Artificial Sequence
  Synthetic nucleic acid aptamer sequence for the B7-HTHC immunocomplex (B7H102)
cctgtcagttgcttaccgacgaaggcgctgaaactgcagcgattcttgcggtgtactgagtgtgtagtgtccttgctcgt

SEQ ID NO: 17 - DNA, 80 bp, Artificial Sequence
  Synthetic nucleic acid aptamer sequence for the B7-HTHC immunocomplex (B7HC1)
cctgtcagttgcttaccggcccaggtgtagtaactgcagggattcttaacgtagctaccgggtgtagtgtccttgctcgt

SEQ ID NO: 18 - DNA, 80 bp, Artificial Sequence
  Synthetic nucleic acid aptamer sequence for the B7-HTHC immunocomplex (B7HC2)
cctgtcagttgcttaccgggcggcacatgctagctgcagcgattcttgtattaggtgcggcgtgtagtgtccttgctcgt

SEQ ID NO: 19 - DNA, 80 bp, Artificial Sequence
  Synthetic nucleic acid aptamer sequence for the B7-HTHC immunocomplex (B7H422)
cctgtcagttgcttaccgcagggcgtcagggacctgcagcgattcttggtgtagggggccgctgtagtgtccttgctcgt

SEQ ID NO: 20 - DNA, 81 bp, Artificial Sequence
  Synthetic nucleic acid aptamer sequence for the B7-HTHC immunocomplex (B7H338)
cctgtcagttgcttaccggcacgggaagttggactgcagcgattctttcgacgcccggccccgtgtagtgtccttgctcgt

SEQ ID NO: 21 - DNA, 79 bp, Artificial Sequence
  Synthetic nucleic acid aptamer sequence derivative for the B7-HTHC immunocomplex (368.2)
cctgtcagttgcttaccgggcacgagtacgagactgcaggattcttctacactgctccccatgtagtgtccttgctcgt

SEQ ID NO: 22 - DNA, 80 bp, Artificial Sequence; identical to SEQ ID NO: 17
  Synthetic nucleic acid aptamer sequence derivative for the B7-HTHC immunocomplex (154)

SEQ ID NO: 23 - DNA, 79 bp, Artificial Sequence
  Synthetic nucleic acid aptamer sequence derivative for the B7-HTHC immunocomplex (444)
cctgtcagttgcttaccgggcacgagtacgagactgcaggattcttctacactgcgccccatgtagtgtccttgctcgt

SEQ ID NO: 24 - DNA, 79 bp, Artificial Sequence
  Synthetic nucleic acid aptamer sequence derivative for the B7-HTHC immunocomplex (489)
cctgtcagttgcttaccgggcacgagtacgagactgcaggattcttctacactggcccccatgtagtgtccttgctcgt

SEQ ID NO: 25 - DNA, 77 bp, Artificial Sequence
  Synthetic nucleic acid aptamer sequence derivative for the B7-HTHC immunocomplex (493)
cctgtcagttgcttaccgggcacgagtacgagactgcaggattcttctacactgcaccccatgtagtgtccttgctc

SEQ ID NO: 26 - DNA, 79 bp, Artificial Sequence
  Synthetic nucleic acid aptamer sequence derivative for the B7-HTHC immunocomplex (815)
cctgtcagttgcttaccgggcacgagtactagactgcaggattcttctacactgccccccatgtagtgtccttgctcgt

SEQ ID NO: 27 - DNA, 79 bp, Artificial Sequence
  Synthetic nucleic acid aptamer sequence derivative for the B7-HTHC immunocomplex (732)
cctgtcagttgcttaccggccacgagtacgagactgcaggattcttctacactgccccccatgtagtgtccttgctcgt

SEQ ID NO: 28 - DNA, 79 bp, Artificial Sequence
  Synthetic nucleic acid aptamer sequence derivative for the B7-HTHC immunocomplex (568)
cctgtcagttgcttaccgggcacgagtacgagactgcaggattcttttacactgccccccatgtagtgtccttgctcgt

SEQ ID NO: 29 - DNA, 79 bp, Artificial Sequence
  Synthetic nucleic acid aptamer sequence derivative for the B7-HTHC immunocomplex (550)
cctgtcagttgcttaccgggcacgagtacgagactgcaggattcttctacactgccccacatgtagtgtccttgctcgt

SEQ ID NO: 30 - DNA, 96 bp, Artificial Sequence
  Synthetic nucleic acid aptamer sequence derivative for the B7-HTHC immunocomplex (333)
cctgtcagttgcttaccgggcacgagtacgagactgcaggattcttctacactgccccccatgtagtgtccttgctcgtgtagtgtccttgctcgt

SEQ ID NO: 31 - DNA, 80 bp, Artificial Sequence
  Synthetic nucleic acid aptamer sequence derivative for the B7-HTHC immunocomplex
cctgtcagttgcttacccggggtggtggctgcgaagccgggattcttaagagggcctgggtgtgtagtgtccttgctcgt

SEQ ID NO: 32 - DNA, 80 bp, Artificial Sequence; identical to SEQ ID NO: 9
  Synthetic nucleic acid aptamer sequence derivative for the B7-HTHC immunocomplex

SEQ ID NO: 33 - DNA, 71 bp, Artificial Sequence
  Synthetic nucleic acid aptamer sequence derivative for the B7-HTHC immunocomplex
cctgtcagttgcttaccgtgtagtgtccttgctcgtcctgtcagttgcttaccgtgtagtgtcctgctcgt

SEQ ID NO: 34 - DNA, 96 bp, Artificial Sequence
  Synthetic nucleic acid aptamer sequence derivative for the B7-HTHC immunocomplex
cctgtcagttgcttaccgggcacgagtacgagactgcaggattcttctacactgccccccatgtagtgtccttcctcgtgtagtgtccttgctcgt

SEQ ID NO: 35 - DNA, 73 bp, Artificial Sequence
  Synthetic nucleic acid aptamer sequence derivative for the B7-HTHC immunocomplex
cctgtcagttgcttaccgtgtagtgtccttgctcgtcctgttcagttgcttaccgtgtagtgtccttgctcgt